(19) United States
(12) Patent Application Publication
(10) Pub. No.: US 2016/0206577 A1
(43) Pub. Date: Jul. 21, 2016

(54) NOVEL DRUG FORMULATION

(71) Applicant: FAB PHARMA SAS, Paris (FR)

(72) Inventors: Vincent GERUSZ, Paris (FR); Coralie MOUZÉ, Paris (FR); Frederic VAN DYCKE, Gent (BE); Dieter AMEYE, Gent (BE)

(21) Appl. No.: 14/914,726

(22) PCT Filed: Aug. 29, 2014
(86) PCT No.: PCT/EP2014/068428

§ 371 Date: Feb. 26, 2016

Related U.S. Application Data
(60) Provisional application No. 61/871,590, filed on Aug. 29, 2013.
(60) Provisional application No. 61/874,516, filed on Sep. 6, 2013.

(30) Foreign Application Priority Data
Jan. 20, 2014 (GB) 1400951.8

(51) Int. Cl. A61K 31/166; A61K 45/06

(52) U.S. Cl. CPC A61K 31/166; A61K 45/06

(57) ABSTRACT

There is provided inter alia a pharmaceutical aqueous nanosuspension comprising (i) 4-(4-ethyl-5-fluoro-2-hydroxyphenoxy)-3-fluorobenzamide in nanoparticulate form and a stabilizing agent.

### FIELD OF THE INVENTION

The present invention relates to novel pharmaceutical nanosuspensions of a known antibacterial agent. The present invention also relates to processes for preparing such nanosuspensions and to their use in the treatment of microbial infections.

### BACKGROUND OF THE INVENTION

The emergence of antibiotic-resistant pathogens has become a serious worldwide healthcare problem as some infections are now caused by multi-drug resistant organisms that are no longer responsive to currently available treatments.

The bacterial fatty acid biosynthesis (FASII system) has generated substantial interest for the development of novel antibacterial and antiparasitic agents (Rock et al. J. Biol. Chem. 2006, 281, 17541; Wright and Reynolds Curr. Opin. Microbiol. 2007, 10, 447). The organization of components in the bacterial fatty acid biosynthesis pathway based on discrete enzymes in some bacteria and parasites is fundamentally different from the multifunctional FASI system found in mammals, therefore allowing good prospects of selective inhibition. The overall high degree of conservation in many enzymes of the bacterial FASII system should also allow the development of broader-spectrum antibacterial and antiparasitic agents.

Among all the monofunctional enzymes of the bacterial FASII system, Fabl represents the enoyl-ACP reductase responsible of the last step of the fatty acid biosynthetic elongation cycle. Using the cofactor NAD(P)H as a hydride source, Fabl reduces the double bond in the trans-2-enoyl-ACP intermediate to the corresponding acyl-ACP product. This enzyme has been shown to constitute an essential target in major pathogens such as E. coli (Heath et al. J. Biol. Chem. 1995, 270, 26538; Bergler et al. Eur. J. Biochem. 1996, 242, 689) and S. aureus (Heath et al. J. Biol. Chem. 2000, 275, 4654, Escaich et al. AAC 2011, 55 (10), 4692, Gerusz et al. JMC 2012, 55, 9914). However, other isoforms have been isolated such as FabK from S. pneumoniae (Heath et al. Nature 2000, 406, 145) and FabL from B. subtilis (Heath et al. J. Biol. Chem. 2000, 275, 40128). Although FabK is structurally and mechanistically unrelated to Fabl (Marrakchi et al. Biochem J. 2003, 370, 1055), the similarity of Fabl with FabL (B. subtilis), InhA (M. tuberculosis) and PfENR (P. falciparum) still offers opportunities of interesting activity spectra (Heath et al. Prog. Lipid Res. 2001, 40, 467).

Several Fabl inhibitors have already been reported in the literature (Tonge et al. Acc. Chem. Res. 2008, 41, 11). Some of them such as diazaborines (Baldock et al. Science 1996, 274, 2107) and isoniazid in its activated form (Tonge et al. Proc. Natl. Acad. Sci. U.S.A. 2003, 100, 13881) act by covalently modifying the cofactor NAD+. However some drawbacks are associated with these products. Diazaborines are only used experimentally because of their inherent toxicity (Baldock et al. Biochem. Pharmacol. 1998, 55, 1541) while isoniazid is a prodrug restricted to the treatment of susceptible tuberculosis. The fact that isoniazid requires activation by hydrogen-peroxide inducible enzymes (Schultz et al. J. Am. Chem. Soc. 1995, 117, 5009) enhances the possibilities of resistance by lack of activation or increased detoxification (Rosner et al. Antimicrob. Agents Chemother. 1993, 37, 2251 and ibid 1994, 38, 1829).

Other inhibitors act by interacting noncovalently with the enzyme-cofactor complex. For instance triclosan, a widely used consumer goods preservative with broad spectrum antimicrobial activity, has been found to be a reversible, tight-binding inhibitor of E. coli Fabl (Ward et al. Biochemistry 1999, 38, 12514). Intravenous toxicology studies on this compound indicated a LD50 on rats of 29 mg/kg clearly ruling out intravenous injection (Lyman et al. Ind. Med. Surg. 1969, 38, 42). Derivatives based on the 2-hydroxydiphenyl ether core of triclosan have been reported (Tonge et al. J. Med. Chem. 2004, 47, 509, ACS Chem Biol. 2006, 1, 43 and Bioorg. Med. Chem. Lett. 2008, 18, 3029; Surolia et al. Bioorg. Med. Chem. 2006, 14, 8086 and ibid 2008, 16, 5536; Freundlich et al. J. Biol. Chem. 2007, 282, 25436) as well as other inhibitors based on various classes of high throughput screening derived templates (Seefeld et al. Bioorg. Med. Chem. Lett. 2001, 11, 2241 and J. Med. Chem. 2003, 46, 1627; Heerding et al. Bioorg. Med. Chem. Lett. 2001, 11, 2061; Miller et al. J. Med. Chem. 2002, 45, 3246; Payne et al. Antimicrob. Agents Chemother. 2002, 46, 3118; Sacchettini et al. J. Biol. Chem. 2003, 278, 20851; Moir et al. Antimicrob. Agents Chemother. 2004, 48, 1541; Montellano et al. J. Med. Chem. 2006, 49, 6308; Kwak et al. Int. J. Antimicro. Ag. 2007, 30, 446; Lee et al. Antimicrob. Agents Chemother. 2007, 51, 2591; Kitagawa et al. J. Med. Chem. 2007, 50, 4710, Bioorg. Med. Chem. 2007, 15, 1106 and Bioorg. Med. Chem. Lett. 2007, 17, 4982; Takahata et al. J. Antibiot. 2007, 60, 123; Kozikowski et al. Bioorg. Med. Chem. Lett. 2008, 18, 3565), nevertheless none of these inhibitors has succeeded yet as a drug. Interestingly, some classes of these inhibitors display activity on both Fabl and FabK: predominantly FabK for the dual compounds based on phenylimidazole derivatives of 4-pyridones (Kitagawa et al. J. Med. Chem. 2007, 50, 4710), predominantly Fabl for the indole derivatives (Payne et al. Antimicrob. Agents Chemother. 2002, 46, 3118; Seefeld et al. J. Med. Chem. 2003, 46, 1627). However, the moderate activity on the second enzyme might prove to be a drawback for such compounds as it may lead to an increase of resistance mechanisms due to the added selection pressure (Tonge et al. Acc. Chem. Res. 2008, 41, 11).

Despite the attractiveness of Fabl as an antibacterial/antiparasitic target, however, it is still largely unexploited at this time.

International application WO 2007/135562 (Mutabilis SA) describes a series of hydroxyphenyl derivatives that display a selective spectrum of activity on species containing Fabl and related targets, in contrast to Triclosan. A specific hydroxyphenyl compound of formula (I) is described in international application WO 2011/026529 (FAB Pharma SAS):

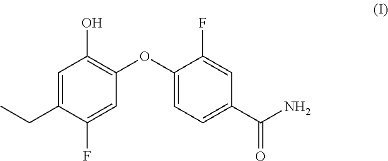 [SMILES: CCc1cc(O)c(Oc2ccc(C(N)=O)cc2F)cc1F]

As described in WO 2011/026529, the compound of formula (I) (referred to herein as “compound (I)”) is known chemically as 4-(4-ethyl-5-fluoro-2-hydroxyphenoxy)-3-fluorobenzamide and has been found to be highly active in vitro against pathogenic methicillin-susceptible Staphylococcus aureus (MSSA), methicillin-resistant Staphylococcus aureus (MRSA), vancomycin-intermediate Staphylococcus aureus (VISA) and vancomycin-resistant Staphylococcus aureus (VRSA) strains. This compound also displays activity against other pathogenic bacteria, including Escherichia coli and Acinetobacter baumannii (Escaich et al. AAC 2011, 55 (10), 4692, Gerusz et al. JMC 2012, 55, 9914). Furthermore, compound (I) is also active in vivo in a murine model against MSSA and MRSA infections. Compound (I) has been evaluated in healthy human volunteers in a Phase I study and was reported to demonstrate ex vivo bactericidal activity against S. aureus.

Compound (I) has relatively poor water-solubility. The injectable pharmaceutical composition comprising compound (I) described in Example 3 of WO 2011/026529 (herein referred to as “the prior art historical formulation”) consists of compound (I), water, glucose monohydrate as a tonicity modifier and hydroxypropyl-beta-cyclodextrin (HPBCD) as a solubilisation agent. Using this formulation, compound (I) may be formulated at concentrations of up to 11.3 mg/mL. However, the use of cyclodextrin in formulations must be carefully considered due to the potential risks of toxicity, as discussed below.

Cyclodextrins are cyclic oligosaccharides made up of six to eight dextrose units (alpha-, beta-, and gamma- “parent” cyclodextrins, respectively) joined though one to four bonds. Cyclodextrins have been used in food and pharmaceutical products for many years, being particularly useful in pharmaceutical compositions due to their ability to form inclusion complexes with drug molecules. The formation of such inclusion complexes can enhance the physicochemical properties of the drug molecule, such as increasing solubility.

On oral administration, only insignificant amounts of intact cyclodextrins are absorbed from the gastrointestinal tract because of their bulky and hydrophilic nature and all of the parent cyclodextrins are accepted as food additives and “generally recognised as safe” (GRAS). In contrast, IV-administered cyclodextrins disappear rapidly from systemic circulation. Following absorption, cyclodextrins distribute to various tissues, although the kidneys have the highest levels and elimination of cyclodextrins has been found to strongly depend on renal clearance (Stella et al. Toxicol. Pathol. 2008, 36, 30). As manifested by a series of changes in the kidney, parenteral administration of alpha-cyclodextrin, beta-cyclodextrin or methylated-beta-cyclodextrin has been observed to result in renal toxicity (Frank et al. Am. J. Pathol. 1976, 83, 367; Irie et al. J. Pharm. Sci. 1997, 86, 147). The maximum reported safe daily dose for HPBCD in commercial intravenous formulations in 16 g/day (Brewster et al. Advanced Drug Delivery Review 2007, 59, 645). As safety is of prime concern when selecting excipients for use in drug formulations, the use of excipients which display any adverse side effects should be avoided where possible.

It is an object of the present invention to provide a pharmaceutically acceptable novel formulation of compound (I) that does not contain HPBCD. Suitably, the novel formulation will potentially be capable of containing a higher concentration of compound (I) compared with the prior art historical formulation, while being resistant to aggregation, thereby having acceptable long term storage stability. Suitably, the novel formulation will have comparable or improved efficacy in vivo, compared with the prior art historical formulation of compound (I).

### SUMMARY OF THE INVENTION

The present inventors have prepared novel pharmaceutical aqueous nanosuspensions of compound (I) which have the advantage of being free from HPBCD and its potential renal toxicity and the practical advantage of allowing compound (I) to be formulated at a much higher concentration compared with the prior art historical formulation. As shown in the Examples, such nanosuspensions are resistant to aggregation, are stable to irradiation, have good long term storage stability, favourable characteristics for administration and suitable efficacy when used in treating bacterial infection.

Thus, in one aspect the present invention provides a pharmaceutical aqueous nanosuspension comprising 4-(4-ethyl-5-fluoro-2-hydroxyphenoxy)-3-fluorobenzamide in nanoparticulate form and a stabilizing agent (hereinafter referred to as “the pharmaceutical nanosuspension of the invention” or “the nanosuspension of the invention”).

### BRIEF DESCRIPTION OF THE FIGURES

FIG. 1 shows an XRPD pattern of a sample of compound (I) prepared using the procedure of Example 1

FIG. 2 shows the particle size distribution (PSD) of a sample of a nanosuspension of the invention prepared by controlled precipitation (Examples 8 and 9)

FIG. 3 shows a thermogravimetric analysis (TGA) of a sample of a nanosuspension of the invention prepared by controlled precipitation (Example 10)

### DETAILED DESCRIPTION OF THE INVENTION

### 4-(4-Ethyl-5-fluoro-2-hydroxyphenoxy)-3-fluorobenzamide (compound (I))

General processes for synthesising compound (I) are disclosed in WO 2007/135562, the contents of which is herein incorporated by reference in its entirety. A specific method for synthesising compound (I) is described in “Example 1 (Alternative procedure)” of WO 2011/026529, the contents of which is also incorporated by reference in its entirety (see also Example 1 of the present application).

The pharmaceutical aqueous nanosuspension of the present invention comprises compound (I) as active ingredient in a therapeutically effective amount. A therapeutically effective amount of compound (I) is defined as an amount sufficient, for a given dose or plurality of divided doses, to achieve a therapeutically meaningful effect in a subject when administered to said subject in a treatment protocol.

In one embodiment, the pharmaceutical aqueous nanosuspension of the invention comprises between about 1 mg/mL to about 1000 mg/mL of compound (I) for example, between about 1 mg/mL to about 500 mg/mL or between about 10 mg/mL and about 100 mg/mL, such as about 90 mg/mL. In another embodiment, the pharmaceutical aqueous nanosuspension of the invention comprises at least 20 mg/mL of compound (I), for example at least 30 mg/mL, at least 40 mg/mL, at least 50 mg/mL, at least 60 mg/mL, at least 70 mg/mL or at least 80 mg/mL of compound (I). The aqueous nanosuspensions of the invention advantageously allows compound (I) to be formulated at a much higher concentration than in the prior art historical formulation.

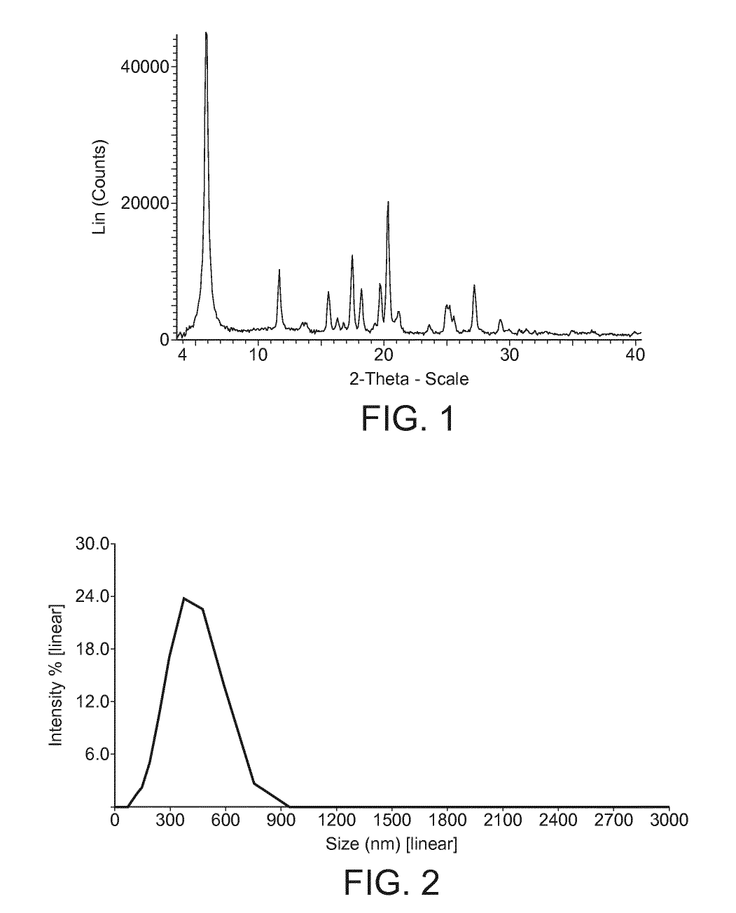

The pharmaceutical aqueous nanosuspension of the invention may be prepared from compound (I) in crystalline form, for example, compound (I) as prepared by a procedure substantially according to Example 1 or Example 2 and/or compound (I) having an XRPD pattern substantially as shown in FIG. 1.

Pharmaceutical Aqueous Nanosuspensions of the Invention

The nanosuspension of the invention comprises nanoparticles of compound (I), defined herein as particles with dimensions of less than 1 μm. A nanosuspension of the invention will suitably have a principal particle size of less than 1 μm i.e. in a particle size analysis (suitably a volume distribution) the largest peak will correspond to a particle diameter of less than 1 μm. A suitable method for analysing particle size is laser diffraction, e.g. using a Mastersizer 2000 instrument from Malvern Instruments or dynamic light scattering, e.g. using a Beckmann Coulter N4 instrument. Laser diffraction may also be used to determine the particle size distribution (PSD) which is usually defined in terms of the volume diameter (Dv). The Dv(0.50), the median, has been defined as the diameter where half of the volume distribution lies below this value. Similarly, 90 percent of the volume distribution lies below the Dv(0.90), and 10 percent of the distribution lies below the Dv(0.10). The term Dv(0.99) may be construed similarly.

The nanosuspension suitably has a volume median diameter (Dv(0.50)) of between about 0.050 μm and about 0.800 μm, for example, between about 0.050 μm and about 0.600 μm, between about 0.100 μm and about 0.500 μm, between about 0.100 μm and about 0.400 μm, between about 0.100 and about 0.300 μm, between about 100 μm and about 200 μm, between about 0.100 μm and about 0.800 μm, between about 0.200 μm and about 0.600 μm, between about 0.300 μm and about 0.500 μm.

The nanosuspension suitably has a Dv(0.10) of between about 0.010 μm and about 0.300 μm, for example, between about 0.010 μm and about 0.100 μm or between about 0.100 μm and about 0.300 μm.

The nanosuspension suitably has a Dv(0.90) of between about 0.300 μm and about 0.900 μm, for example, between about 0.300 μm and about 0.500 μm or between about 0.500 μm and about 0.900 μm.

The present inventors have discovered that a stable aqueous nanosuspension of compound (I) in nanoparticulate form may be prepared without the use of HPBCD as a solubilisation agent. Thus, in one embodiment, the pharmaceutical aqueous composition of the invention is substantially free of cyclodextrin, in particular HPBCD.

A known drawback of drug nanosuspensions is their limited long term stability due to, inter alia, settling and Ostwald-ripening effects. The aqueous nanosuspensions of the present invention were found to exhibit good long term storage stability.

The aqueous nanosuspension of the invention comprises a stabilizing agent to stabilize the nanosuspension by preventing agglomeration of the nanoparticles in the solution and by preventing or minimising the formation of large particles i.e. particles with dimensions >1 μm. Examples of such stabilizing agents are well known to a person of skill in the art. Suitably the stabilizing agent is a surfactant or surfactant polymer. Suitably the stabilizing agent is water soluble. Suitable surfactants for use in the nanosuspension of the invention include, but are not limited to, polysorbate surfactants, poloxamer surfactants, dioctyl sodium sulfosuccinate (DOSS). A typical polysorbate surfactant is Tween®, for example Tween 20® or Tween 80®. Typical poloxamer surfactants include poloxamer 188 and poloxamer 228. Polyvinylpyrrolidone (also known as Povidone or PVP) is a water soluble polymer made from the monomer of N-vinylpyrrolidone. A suitable surfactant polymer is polyvinylpyrrolidone (PVP). PVP is often defined in terms of a K-value which characterises the mean molecular weight e.g. Povidone K 12, Povidone K 17, Povidone K 25, Povidone K 30 and Povidone K 90. PVP is available under various trade names including Plasdone C-15®, Kollidon 12PF®, Kollidon 17PF® and Kollidon 30®. In one embodiment, the PVP has a mean molecular weight of between about 2,000 Da and 1,500,000 Da, such as between about 2,000 Da and about 5,000 Da; between about 6,000 Da and about 12,000 Da; between about 25,000 Da and about 40,000 Da; between about 41,000 Da and about 65,000 Da or between about 1,000,000 Da and about 1,500,000 Da. Suitably, the PVP has a mean molecular weight between about 2,000 Da and about 3000 Da (corresponding to Kollidon 12).

Preferably the stabilizing agent is PVP. PVP is widely used in pharmaceutical preparations and is well tolerated.

The nanosuspension of the invention will contain a stabilizing agent in an amount which is sufficient to provide acceptable long term storage stability of the nanosuspension i.e. will prevent agglomeration and/or prevent/minimise the formation of particles with dimensions >1 μm upon storage. For example, typically the stabilising agent will stabilise the nanosuspension such that the principal peak in the particle size distribution is maintained at a particle size diameter of <1 μm, such as <0.800 μm, <0.700 μm, <0.600 μm, or <0.500 μm for at least 2 months, for example at least 3 months, at least 6 months, at least 9 months, at least 12 months or at least 24 months when stored under conditions of 5° C./ambient relative humidity, or conditions of 25° C./60% relative humidity. The exact amount of stabilising agent in the nanosuspension will depend on the concentration of compound (I) and on the particular stabilising agent used.

In one embodiment, the stabilising agent is present in the nanosuspension at a concentration of between about 1 mg/mL and about 1000 mg/mL, for example between about 1 mg/mL and about 500 mg/mL, between about 1 mg/mL and about 250 mg/mL, between about 10 mg/mL and about 100 mg/mL, or between about 20 mg/mL and 75 mg/mL.

In another embodiment, the stabilising agent is PVP and is present in the nanosuspension at a concentration of between about 1 mg/mL and about 500 mg/mL, for example between about 1 mg/mL and about 250 mg/mL, between about 10 mg/mL and about 100 mg/mL, or between about 20 mg/mL and 75 mg/mL, such as about 50 mg/mL.

Attempts to prepare nanoparticles of compound (I) by solvent evaporation, solvent injection and the reconstitution of spray dried powders were found to be unsuitable due to the formation of large, non-nanoparticulate particles of compound (I). Alternative solvent evaporation systems such as oil-in-oil and water-in-oil systems were investigated, however large particle sizes were still observed. In an attempt to reduce solvent dissolution into the continuous phase of the oil-in-water system, the aqueous phase was saturated with different salts to reduce solvent solubility. However, little benefit was achieved through the use of the saturated salt systems, as large crystals were still observed. It was thought that the use of a concentrated aqueous polymer solution would disrupt or prevent crystal formation. Multiple molecular weights and concentrations of PVP were studied. A low molecular weight PVP (3,500 Da) was found to be suitable for making suspensions with 4.4 μm particles of compound (I). In the expectation of further reducing the particle size (to <1 μm i.e. to nanoparticulate form) further surfactants were added. However, by contrast, the addition of further surfactants to the solution yielded large size growth of particles.

Further experimentation by the inventors revealed that nanoparticles of compound (I) could be prepared using wet milling method or controlled precipitation methods in the presence of a stabilizing agent and water, to yield a stable aqueous nanosuspension of compound (I). Given the previous failures of solvent evaporation and other methods, with or without surfactant as a stabilising agent, this was unexpected. Furthermore, the nanosuspensions prepared by wet milling and controlled precipitation allowed compound (I) to be formulated at a much higher concentration than the prior art historical formulation, as discussed above.

Preparation of a Pharmaceutical Aqueous Nanosuspension of the Invention by Wet Milling

In one embodiment there is provided a process for preparing a pharmaceutical aqueous nanosuspension comprising the steps of:

(a) preparing an aqueous suspension comprising particulate 4-(4-ethyl-5-fluoro-2-hydroxyphenoxy)-3-fluorobenzamide and a stabilizing agent; and
(b) milling the aqueous suspension of step (a) to form nanoparticulate 4-(4-ethyl-5-fluoro-2-hydroxyphenoxy)-3-fluorobenzamide.

A representative wet milling procedure is set out in Example 3. The nanosuspension is prepared by first suspending particulate compound (I) in an aqueous solution comprising a stabilizing agent. The aqueous suspension is then subjected to a milling step in order to reduce the particle size of compound (I) material such that the principal peak in a particle size distribution will correspond to a particle diameter of less than 1 μm, or until the target particle size distribution is reached.

In this embodiment, typically the stabilizing agent is a surfactant or a surfactant polymer. Suitably, the stabilizing agent is PVP. In step (a) the stabilizing agent is usually dissolved in the water prior to the addition of compound (I). When compound (I) is added to the dissolved solution of stabilizing agent a suspension is formed, due to the insolubility/partial insolubility of compound (I) in water. The suspension is then suitably transferred to a glass vial in preparation of the milling step (b).

The step of milling (step (b)), for example, involves the addition of milling media to the vial, which is then placed on a roller mill. It is also possible to vary the order of steps e.g. to first add the milling media to the vial, then to add compound (I) and then the solution of stabilizing agent. Based on the compound (I) particle size to be achieved and the size of the vial, suitable milling media may be selected. Typical milling media comprise beads of ceramics (cerium or yttrium-stabilised zirconium oxide), stainless steel, glass or highly cross-linked polystyrene resin, all of which can be obtained with various diameters, typically between 0.1-1 mm. Suitably the milling media are milling beads made from yttrium-stabilised zirconium oxide with diameter 0.5 mm. An alternative diameter is 1.0 mm. The wet milling step may be carried out using any suitable apparatus, such as a roller mill or a rotor-stator wet mill.

In one embodiment, the compound (I) starting material used in step (a) is prepared substantially according to the procedure of Example 1 or Example 2. In this embodiment, the compound (I) starting material has an XRPD pattern substantially as shown in FIG. 1.

In one embodiment, compound (I) is added at a concentration of between about 1 mg/mL to about 500 mg/mL, for example between about 10 mg/mL and about 100 mg/mL, such as about 90 mg/mL or between about 10 mg/mL and about 400 mg/mL e.g. between about 100 mg/mL and about 300 mg/mL. As the composition of the starting suspension (i.e. prior to wet milling) does not change during the milling process, the concentration of compound (I) in the nanosuspension may be expected to remain the same (within experimental error) throughout the milling process and in the final nanosuspension.

In one embodiment, the stabilising agent is PVP and is added at a concentration of between about 10 mg/mL and about 100 mg/mL, for example between about 20 mg/mL and 75 mg/mL, such as about 50 mg/mL or between about 50 mg/mL and 200 mg/mL e.g. between about 50 mg/mL and 150 mg/mL.

As the composition of the starting suspension (i.e. prior to wet milling) does not change during the milling process, the concentration of PVP (or more generally, the concentration of the stabilising agent) in the nanosuspension is expected to remain roughly the same throughout.

Suitably, both steps (a) and (b) are carried out at room temperature.

Step (b) i.e. the milling process, is carried out for as long as required to reach the desired particle size. For example, milling step (b) may be carried out until at least 90% of the suspended particles are smaller than 1.000 μm, 0.900 μm, 0.800 μm, 0.700 μm, 0.600 μm or 0.500 μm or until the target particle size distribution is reached.

In one embodiment, there is provided a pharmaceutical aqueous nanosuspension obtainable by wet milling a suspension of particulate 4-(4-ethyl-5-fluoro-2-hydroxyphenoxy)-3-fluorobenzamide and stabilizing agent in water.

The particle size distributions (PSDs) of three different batches of nanosuspension prepared by the wet milling procedure described in Example 3 were analysed and the results shown in Example 4. The PSDs were as follows: Dv(0.10)=0.066-0.067 μm; Dv(0.50)=0.133-0.134 μm; and Dv(0.90)=0.314-0.358 μm, firstly indicating that the procedure formed the desired size of particle (i.e. nanoparticles), and secondly indicating the reproducibility of the procedure, as a very similar particle size distribution was observed in each batch.

Nanosuspensions prepared by the wet milling method of Example 3 were stored at different temperatures and relative humidities for different lengths of time in order to assess the storage stability of the nanosuspension. The results are shown in Example 5, where it can be seen that the nanosuspension exhibited good stability under typical storage conditions and “accelerated” conditions of relatively higher temperature and relative humidity.

Preparation of a Pharmaceutical Aqueous Nanosuspension of the Invention by Controlled Precipitation

In this embodiment, there is provided a process for preparing a pharmaceutical aqueous nanosuspension comprising the steps of:

(i) dissolving 4-(4-ethyl-5-fluoro-2-hydroxyphenoxy)-3-fluorobenzamide in an organic solvent;
(ii) adding the solution of step (i) to a solution of a stabilizing agent dissolved in water, whilst maintaining a homogenous solution;
(iii) stirring the solution of step (ii); and
(iv) slowly adding the solution of step (iii) to cool water to form 4-(4-ethyl-5-fluoro-2-hydroxyphenoxy)-3-fluorobenzamide in nanoparticulate form.

A representative controlled precipitation procedure is set out in Example 8.

The organic solvent of step (i) is one in which compound (I) dissolves completely, for example ethyl acetate. In one embodiment, in step (a) compound (I) is dissolved in organic solvent to a concentration of between about 10 mg/mL and 200 mg/mL, for example between about 50 mg/mL and about 100 mg/mL.

Suitably the stabilizing agent is PVP. When the solution of step (i) is combined with water (i.e. step (ii)), compound (I) remains fully dissolved, in a homogenous solution. In one embodiment, the stabilising agent is a surfactant or surfactant polymer. Suitably, the stabilising agent is PVP dissolved in water, suitably at a concentration of about 40% w/w.

Typically, step (iii) takes place at a temperature of between about 30° C. and about 50° C., suitably about 40° C. For example, the solution of step (ii) is stirred for 4 h at 40° C. before being stirred overnight at room temperature.

Typically, step (iv) takes place in cool water. “Cool” water in this case is water which is at a sufficiently low temperature to induce precipitation of compound (I), typically between about 0° C. and about 5° C., suitably about 2° C. The water added in step (ii) is not cool water, i.e. when combined with the solution of step (i) it does not cause precipitation of compound (I), which remains in solution. Typically the water added in step (ii) is at ambient temperature, for example room temperature, such as about 21° C. or above. While the solution of step (iii) is added to the cool water, the cool water will be stirred, suitably at a rate of about 600 rpm. Suitably, the solution of step (iii) will be pumped into the cool water at a rate of 10 ml/min. Suitably, the solution will be pumped under the surface of the cooled water.

After the solution of step (iii) has been added, the combined compound (0/organic solvent/water/stabilizing agent solution will be hazy, due to the formation of nanoparticles of compound (I). Suitably, this hazy solution (the nanosuspension) will be cooled and stirred overnight, before being allowed to warm to room temperature.

The nanosuspension of step (iv) may be washed with water and concentrated, for example using a cross flow system e.g. PureTec Autopurification system. Excess stabilizing agent such as PVP may be removed via cross flow filtration.

In one embodiment, the compound (I) starting material used is prepared substantially according to the procedure of Example 1 or Example 2. In this embodiment, the compound (I) starting material has an XRPD pattern substantially as shown in FIG. 1.

In one embodiment, there is provided a pharmaceutical aqueous nanosuspension comprising 4-(4-ethyl-5-fluoro-2-hydroxyphenoxy)-3-fluorobenzamide and a stabilizing agent obtainable by controlled precipitation of nanoparticles of 4-(4-ethyl-5-fluoro-2-hydroxyphenoxy)-3-fluorobenzamide.

The particle size distribution of the nanosuspension prepared by the controlled precipitation procedure described in Example 8 was measured as described in Example 9, and the nanosuspension was found to have a mean particle size (Dv(0.50)) of 391.5 nm (intensity %) measured using dynamic light scattering, without any microparticles (i.e. particles >1 μm). Zeta potential analysis indicated the good potential stability of this nanosuspension (Example 11).

Pharmaceutical Uses and Methods of Administration

As illustrated in the Examples, the aqueous nanosuspensions of the invention have potential therapeutic utility, in particular for treating infection by microbial pathogens.

In one embodiment, there is provided the pharmaceutical aqueous nanosuspension, as described herein, for use in treating microbial infection in humans and animals.

In a further embodiment, the present invention provides the use of pharmaceutical aqueous nanosuspension, as described herein, for the manufacture of a medicament for the treatment of microbial infection.

In a still further embodiment, the present invention provides a method of treating microbial infection comprising administering to a subject an effective amount of a pharmaceutical aqueous nanosuspension, as described herein.

Pharmaceutical aqueous nanosuspensions of the present invention are particularly useful in forming a therapeutic regimen having a selective spectrum of activity in vitro and in vivo against bacterial strains which are susceptible to Fabl inhibition.

Such strains encompass Staphylococcus aureus including multiresistant strains (such as methicillin-susceptible Staphylococcus aureus (MSSA), methicillin-resistant Staphylococcus aureus (MRSA), vancomycin-intermediate Staphylococcus aureus (VISA) and vancomycin-resistant Staphylococcus aureus (VRSA) strains), Acinetobacter strains (such as Acinetobacter baumannii), Bacillus anthracis, Chlamydophila pneumoniae, Escherichia coli, Haemophilus influenzae, Helicobacter pylori, Klebsiella pneumoniae, Neisseria meningitidis, Neisseria gonorrhoeae, S. intermedius, P. multocida, B. bronchiseptica, M. haemolytica and A. pleuropneumoniae and also bacteria such as Mycobacterium tuberculosis carrying homologous Fabl enzymes such as InhA or other organisms such as Plasmodium falciparum.

According to the European Pharmacopeia (Eur. Ph.) osmolality of 270-330 mOsm/kg is recommended for a parenteral isotonic solution/suspension, therefore in embodiments of the invention where the osmolality of the nanosuspension of the invention is below or above this recommended range, then the nanosuspension can be further formulated for an ex-tempore use in a pharmaceutical composition. Thus, in one embodiment, the nanosuspension of the invention additionally comprises a tonicity modifier. Suitable tonicity modifiers include, but are not limited to, glucose, mannitol and NaCl, which are typically dissolved in water to form a stock solution before being added to the nanosuspension. An isotonic nanosuspension suitable for intravenous (IV) administration will typically have osmolality of 250-350 mOsm/kg, for example 280-320 mOsm/kg. Use of glucose as tonicity modifier appears to result in more stable nanosuspensions than use of NaCl (see Examples 7A, 7B, 14 and 14A). Thus use of an uncharged tonicity modifier (such as glucose or mannitol, preferably glucose) appears to be preferred.

Thus, in one embodiment, an aqueous nanosuspension of the invention further comprises a tonicity modifier and has osmolality between about 250 mOsm/kg and about 350 mOsm/kg, such as between about 280 mOsm/kg and about 320 mOsm/kg.

Examples 6, 6A and 6B provide specific examples of isotonic formulations which may be prepared from the concentrated nanosuspensions of the invention and are suitable for IV administration. The short term stability of the isotonic formulations was assessed. As shown in Examples 7, 7A and 7B, the formulations described in Example 6, 6A and 6B are suitable for an ex-tempore IV administration since the osmolarity, pH and particle size distribution are relatively stable over 48-69 hours and indeed, upon longer term storage, up to 6 weeks. As shown in Examples 14 and 14A, the formulations described in Examples 6A and 6B are stable to irradiation by gamma rays.

Pharmaceutical aqueous nanosuspensions of the present invention may, for example, be formulated with a pH of 3-9 e.g. 4-8 when measured at 21° C. By way of non-limiting example, acetate buffer may usefully be employed to buffer between around pH 3.75 and 5.75 (e.g. between pH 4.2 and 5.3).

The nanosuspension of the invention may comprise one or more medicaments in addition to compound (I). Suitably, the additional medicament is an antibacterial agent. Thus in one embodiment, the nanosuspension of the invention additionally comprises an antibacterial agent selected from the group consisting of carbapenems, aminoglycosides, polymyxins, glycylcyclines, rifampicin and sulbactam.

Examples of carbapenems include but are not limited to: meropenem, imipenem, ertapenem, doripenem, panipenem and biapenem. In one embodiment, the carbapenem is meropenem or imipenem, suitably meropenem.

Examples of aminoglycosides include but are not limited to amikacin, gentamicin, tobramycin, arbekacin, kanamycin, neomycin, netilmicin, paromomycin, rhodostreptomycin, streptomycin and apramycin. In one embodiment, the aminoglycoside antibacterial agent is amikacin, gentamicin or tobramycin, suitably amikacin.

In one embodiment, the polymyxin antibacterial agent is colistin, suitably colistin methanesulfonate sodium.

In one embodiment, the glycylcycline antibacterial agent is tigecycline.

In one embodiment, the additional medicament is an antibacterial agent selected from the group consisting of meropenem, imipenem, amikacin, gentamicin, tobramycin, colistin, tigecycline, rifampicin and sulbactam and is suitably selected from the group consisting of meropenem, amikacin and colistin.

Nanosuspensions of the invention have comparable efficacy in vivo compared to the prior art historical formulation. As shown in Example 16, a pharmaceutical aqueous nanosuspension of the invention prepared according to Example 3 showed comparable survival percentages against S. aureus in a murine systemic model of infection as the prior historical formulation of compound (I) comprising HPBCD. As shown in Example 17, a pharmaceutical aqueous nanosuspension of the invention prepared according to Example 3 also showed comparable efficacy against MSSA and MRSA compared with the prior art historical formulation of compound (I) in a neutropenic mouse thigh infection model.

As described in Example 18, a tissue distribution study was conducted with the aim of comparing the PK profile in rat plasma, liver, heart, lungs, skin and bone of a pharmaceutical aqueous nanosuspension of the invention with the prior art historical formulation. A higher exposure in plasma and tissues was observed for the prior art historical formulation compared with the pharmaceutical aqueous nanosuspensions of the invention as the plasma clearance and the volume of distribution of the prior art historical formulation was halved compared with the pharmaceutical aqueous nanosuspensions of the invention, resulting in greater plasma exposure.

However, despite the nanosuspensions of the invention having a lower plasma concentration that the prior art historical formulation, the tissue-plasma partition coefficient was found to be higher for the pharmaceutical aqueous nanosuspension of the invention in all matrices when compared with the prior art historical formulation. This implies that the pharmaceutical aqueous nanosuspensions of the invention provide better penetration into the tissues than the prior art historical formulation, thereby allowing a lower concentration of formulation to be administered.

Suitably, the pharmaceutical aqueous nanosuspension of the invention is administered parenterally, e.g. intravenously or subcutaneously or intra-peritoneally. Thus suitably the pharmaceutical aqueous nanosuspension of the invention is a pharmaceutical aqueous parenteral nanosuspension.

Typically, the dosage of compound (I) as a pharmaceutical aqueous nanosuspension to be administered to the subject will be between about 1000 mg/kg and about 0.1 mg/kg, such as between about 800 mg/kg and about 1 mg/kg, between about 500 mg/kg and about 10 mg/kg or between about 300 mg/kg and about 100 mg/kg, such as about 200 mg/kg.

Pharmaceutical aqueous nanosuspensions of the present invention provide an advantageous alternative to the prior art formulation of compound (I), as the nanosuspensions of the present invention do not comprise HPBCD, therefore avoid the potential toxicity issues with the cyclodextrin while allowing compound (I) to be formulated at a much higher concentration than in the prior art formulation. As demonstrated in Example 5, nanosuspensions of the present invention have good long term stability while being stored for up to 9 months. Furthermore, ex-tempore formulations (with suitable osmolality for parenteral administration) were found to have suitable short term stability. In vivo, nanosuspensions of the invention have been found to have comparable efficacy when compared to the prior art historical formulation with a rat PK profile that suggests that nanosuspensions of the invention have better penetration into the tissue.

In one embodiment of the invention, the pharmaceutical aqueous nanosuspension comprises PVP as a stabilising agent. PVP as an additive provides a significant advantage over the HPBCD used in the currently known formulation, due to its non-toxic properties. Specifically, PVP has good tolerance and low molecular weight grades such as K12 are quickly eliminated from the system and have already been used in parenteral formulations (http://www.pharma-ingredients.basf.com/Statements/Technical%20Informations/EN/Pharma%20Solutions/03_0307 30e_Soluble%20Kollidon%20grades.pdf).

### FURTHER EMBODIMENTS OF THE INVENTION

In one embodiment, there is provided a pharmaceutical aqueous nanosuspension comprising 4-(4-ethyl-5-fluoro-2-hydroxyphenoxy)-3-fluorobenzamide in nanoparticulate form and a stabilizing agent, wherein the 4-(4-ethyl-5-fluoro-2-hydroxyphenoxy)-3-fluorobenzamide is present in the nanosuspension at a concentration of at least 80 mg/mL, and wherein the stabilising agent is PVP.

In one embodiment, there is provided a pharmaceutical aqueous nanosuspension comprising 4-(4-ethyl-5-fluoro-2-hydroxyphenoxy)-3-fluorobenzamide in nanoparticulate form and a stabilizing agent, wherein the volume median diameter (Dv(0.50)) of the nanosuspension is between about 0.050 μm and 0.500 μm, and wherein the stabilising agent is PVP.

In one embodiment, there is provided a pharmaceutical aqueous nanosuspension comprising 4-(4-ethyl-5-fluoro-2-hydroxyphenoxy)-3-fluorobenzamide in nanoparticulate form and a stabilizing agent, wherein the volume median diameter (Dv(0.50)) of the nanosuspension is between about 0.050 μm and 0.500 μm, wherein the 4-(4-ethyl-5-fluoro-2-hydroxyphenoxy)-3-fluorobenzamide is present in the nanosuspension at a concentration of at least 80 mg/mL, and wherein the stabilising agent is PVP.

In one embodiment, there is provided a process for preparing a pharmaceutical aqueous nanosuspension comprising the steps of:

(a) preparing an aqueous suspension comprising particulate 4-(4-ethyl-5-fluoro-2-hydroxyphenoxy)-3-fluorobenzamide and a stabilising agent; and
(b) milling the aqueous suspension of step (a) to form nanoparticulate 4-(4-ethyl-5-fluoro-2-hydroxyphenoxy)-3-fluorobenzamide,
wherein the stabilising agent is PVP and wherein the resulting 4-(4-ethyl-5-fluoro-2-hydroxyphenoxy)-3-fluorobenzamide in nanoparticulate form has a volume median diameter (Dv0.5) from about 0.050 μm to about 0.600 μm.

In one embodiment, there is provided a process for preparing a pharmaceutical aqueous nanosuspension comprising the steps of:

(a) preparing an aqueous suspension comprising particulate 4-(4-ethyl-5-fluoro-2-hydroxyphenoxy)-3-fluorobenzamide and a stabilising agent; and
(b) milling the aqueous suspension of step (a) to form nanoparticulate 4-(4-ethyl-5-fluoro-2-hydroxyphenoxy)-3-fluorobenzamide,
wherein the stabilising agent is PVP, and wherein the 4-(4-ethyl-5-fluoro-2-hydroxyphenoxy)-3-fluorobenzamide of step (a) is prepared substantially according the procedure of Example 1 or Example 2.

In one embodiment, there is provided a process for preparing a pharmaceutical aqueous nanosuspension comprising the steps of:

(a) preparing an aqueous suspension comprising particulate 4-(4-ethyl-5-fluoro-2-hydroxyphenoxy)-3-fluorobenzamide and a stabilising agent; and
(b) milling the aqueous suspension of step (a) to form nanoparticulate 4-(4-ethyl-5-fluoro-2-hydroxyphenoxy)-3-fluorobenzamide,
wherein the stabilising agent is PVP, and wherein the 4-(4-ethyl-5-fluoro-2-hydroxyphenoxy)-3-fluorobenzamide of step (a) is has an XRPD pattern substantially as shown in FIG. 1.

In one embodiment, there is provided a process for preparing a pharmaceutical aqueous nanosuspension comprising the steps of:

(i) dissolving 4-(4-ethyl-5-fluoro-2-hydroxyphenoxy)-3-fluorobenzamide in an organic solvent;
(ii) adding the solution of step (i) to a solution of a stabilizing agent dissolved in water, whilst maintaining a homogenous solution;
(iii) stirring the solution of step (ii); and
(iv) slowly adding the solution of step (iii) to cool water to form 4-(4-ethyl-5-fluoro-2-hydroxyphenoxy)-3-fluorobenzamide in nanoparticulate form,
wherein the stabilising agent is PVP and wherein the resulting 4-(4-ethyl-5-fluoro-2-hydroxyphenoxy)-3-fluorobenzamide in nanoparticulate form has a volume median diameter (Dv(0.5)) from about 0.050 μm to about 0.600 μm.

In one embodiment, there is provided a process for preparing a pharmaceutical aqueous nanosuspension comprising the steps of:

(i) dissolving 4-(4-ethyl-5-fluoro-2-hydroxyphenoxy)-3-fluorobenzamide in ethyl acetate;
(ii) adding the solution of step (i) to a solution of PVP dissolved in water, whilst maintaining a homogenous solution;
(iii) stirring the solution of step (ii) at a temperature of between about 30° C. and about 50° C.; and
(iv) slowly adding the solution of step (iii) to water at a temperature of between about 0° C. and about 5° C. to form 4-(4-ethyl-5-fluoro-2-hydroxyphenoxy)-3-fluorobenzamide in nanoparticulate form,
wherein the stabilising agent is PVP and wherein the resulting 4-(4-ethyl-5-fluoro-2-hydroxyphenoxy)-3-fluorobenzamide in nanoparticulate form has a volume median diameter (Dv(0.5)) from about 0.050 μm to about 0.600 μm.

In one embodiment, there is provided a pharmaceutical aqueous nanosuspension of the invention which is stable to storage at 25° C. and 60% RH for up to 6 weeks. By “stable to storage” means that the Dv(0.90) of the nanosuspension does not exceed 1 μm, preferably does not exceed 0.9 μm or more preferably 0.8 μm e.g. does not exceed 0.6 μm after storage in a closed vial at 25° C. and 60% RH for up to 6 weeks (see methods of Examples 7A and 7B).

It may be suitable to irradiate the pharmaceutical aqueous nanosuspension of the invention with gamma rays of up to 40 kGy e.g. in order to ensure sterility prior to storage.

In one embodiment, there is provided a pharmaceutical aqueous nanosuspension of the invention which is stable to irradiation with gamma rays of up to 40 kGy. By “stable to irradiation with gamma rays of up to 40 kGy” means that the Dv(0.50) and further preferably the Dv(0.90) of the nanosuspension does not exceed 1 μm, preferably does not exceed 0.9 μm or more preferably 0.8 μm e.g. does not exceed 0.6 μm after irradiation with gamma rays of up to 40 kGy (see methods of Examples 12, 13, 14 and 14A). Preferably the Dv(0.50) and further preferably the Dv(0.90) of the nanosuspension does not exceed 1 μm, preferably does not exceed 0.9 μm or more preferably 0.8 μm e.g. does not exceed 0.6 μm after irradiation with gamma rays of up to 40 kGy and subsequent storage in a closed vial at 25° C. and 60% RH for up to 6 weeks (see methods of Examples 14 and 14A).

### ABBREVIATIONS

CFU colony forming units
hr hours
HPBCD hydroxypropyl-beta-cyclodextrin
IV intravenous
min minutes
MSSA methicillin-susceptible Staphylococcus aureus
MRSA methicillin-resistant Staphylococcus aureus
PK pharmacokinetic
PVP polyvinylpyrrolidone
RH relative humidity
TFA trifluoroacetic acid

### EXAMPLES

### General Procedures

All starting materials and solvents were obtained either from commercial sources or prepared according to the procedure described herein, or described in the literature citation. Unless otherwise stated all reactions were stirred. Organic solutions were routinely dried over anhydrous magnesium sulfate.

Analytical Methods

X-Ray Powder Diffraction (XRPD)

XRPD analysis was performed on a Brüker-AXS D8 Advance diffractometer, using a copper anti-cathode, a mono-crystalline silicon sample holder and a position sensitive detector. Powder sample was dispersed on the silicon sample holder in a way to avoid preferred orientation (not randomly oriented crystals) and to ensure planarity of the specimen surface. Instrument operating conditions for X-ray pattern acquisition are as follows: temperature: ambient; atmosphere: ambient; X-ray generator: 40 kV at 40 mA; X-ray source: Cu target; emission radiation Kλ1 (nm)=0.15406; Kλ2 (nm)=0.15444; ratio Kλ2/Kλ1=0.5; Kβ filter radiation=nickel; slit (anti-divergence)=0.6; goniometer: (angular sector analyzed)=3.5-40 or 3.5-70 (° for 20); step size=0.069 (° for 20); rotation speed for sample holder: 30 rpm; detection: angular opening=8°; step time for measuring diffracted intensity=6 s.

HPLC Analysis (Amount of Compound (I) in Nanosuspensions)

The following HPLC method was used to determine the amount of compound (I) in the nanosuspensions of the invention.

Instrument:

HPLC system with UV detector; column: Waters X-Bridge Shield RP18 3.5 μm, 150 mm×3.0 mm; mobile phase: A: ultrapure water (milliQ or equivalent)+0.05% TFA; B: acetonitrile+0.05% TFA; run time: 28 min; wavelength: 282 nm; flow rate: 0.70 mL/min; column temperature: 50° C.; autosampler temperature: 20±5° C.; injection volume: 25 μl.

TABLE 1
Chromatographic condition:

| Time (min) | Flow (mL/min) | % A | % B |
|---|---|---|---|
| 0.0 | 0.70 | 75.0 | 25.0 |
| 2.0 | 0.70 | 75.0 | 25.0 |
| 12.0 | 0.70 | 30.0 | 70.0 |
| 15.0 | 0.70 | 30.0 | 70.0 |
| 18.0 | 0.70 | 75.0 | 25.0 |
| 28.0 | 0.70 | 75.0 | 25.0 |

Laser Diffraction Analysis (Nanosuspension Particle Size Distribution—Example 4)

Particle size distribution of the suspended particles was determined using laser diffraction, type Mastersizer 2000 (Malvern, Worcestershire, UK). A Hydro 2000S sample dispersion module was used. The sample holder was filled with purified water, an aliquot of the nanosuspension was dispersed in the sample holder at 1500 rpm. Each analysis was performed in duplicate. The particle size is expressed via the volume diameter (Dv) using the Malvern Software. The Dv(0.50), the median, has been defined as the diameter where half of the volume distribution lies below this value. Similarly, 90 percent of the volume distribution lies below the Dv(0.90), and 10 percent of the distribution lies below the Dv(0.10).

Dynamic Light Scattering (Nanosuspension Particle Size Analysis—Example 9)

The size of the suspended particles was analysed using a Beckman Coulter N4 Plus, which is a photon correlation spectrometer that measures particle size based on the principles of dynamic light scattering. This method provides an intensity % value rather than a volume %. However, this data can be manipulated to give approximate Dv(0.10), Dv(0.50) and Dv(0.90) values.

Osmolality Measurement

Osmolality was measured using an Advanced Micro Osmometer (Model 3320 Advanced Instruments Inc, Norwood, Mass., US) by the freezing-point method. Clinitrol™ 290 was used as reference solution (Advanced Instruments). Results are represented as mOsm/kg. According the Eur. Ph., 270-330 mOsm/kg is recommended for a parenteral isotonic solution/suspension.

Zeta Potential (Electrostatic Stability)

Zeta potential was measured using a PALS Zeta Potential Analyzer Ver. 3.40 (Brookhaven Instruments Corp.) using the following parameters.

TABLE 2
Zeta potential parameters
Instrument parameters

| Sample Count | 788 | kcps | Cycles per run | 50 | |
|---|---|---|---|---|---|
| Rate | | | | | |
| Ref. Count Rate | 1690 | kcps | Voltage | 4.00 | Volts |
| Wavelength | 659.0 | nm | Electric Field | 10.01 | V/cm |

| | | |
|---|---|---|
| Field Frequency | 2.00 | Hz |

Measurement parameters

| Zeta Potential | Smoluchowski | | Temperature | 25.0° | C. |
|---|---|---|---|---|---|
| Model | | | | | |
| Mean Mobility | −3.39 | (μ/s)/(V/cm) | Viscosity | 0.890 | cP |
| pH | 4.41 | | Refractive Index | 1.330 | |
| Conductance | 11 | μS | Dielectric | 78.54 | |
| | | | Constant | | |
| Concentration | 2.00 | mg/mL | Particle Size | 160.0 | nm |

| | |
|---|---|
| Liquid | Water |

### Example 1

### Preparation of 4-(4-ethyl-5-fluoro-2-hydroxyphenoxy)-3-fluorobenzamide (compound (I))

Compound (I) may be prepared using “Example 1 (Alternative Procedure)” of WO 2011/026529 (page 18 line 4 though to page 19 line 6). Material isolated using this procedure was found to be crystalline. An XRPD pattern obtained from a sample of compound (I) prepared substantially according to this procedure is shown in FIG. 1.

### Example 2

### Crystallization of Compound (I)

Compound (I) in crystalline form having an XRPD pattern substantially as shown in FIG. 1 may also be prepared using the following crystallization procedure. Compound (I) (600 mg) was dissolved under magnetic stirring at 90° C. (reflux) in 24 mL of toluene/EtOH (95/5 v/v). The reaction mixture was stirred at 90° C. for 1 h and then cooled by decreasing the temperature by 5° C. every 10 min (30° C. per hour). Crystallization observed at 70° C. and reaction mixture cooled to 25° C. (5° C. every 10 min). The resulting solid was filtered at 25° C. and dried under reduced pressure at 80° C. for 1 h to yield Compound (I) having an XRPD pattern substantially as shown in FIG. 1 (80% yield).

### Example 3

### Preparation of Nanosuspension of the Invention by Wet Milling

A nanosuspension of compound (I) was prepared by wet milling as follows. The milling medium was prepared by dissolving a polymer, Kollidon 12PF® (PVP; 50.0 mg/mL) in purified water (50 mL). The solution was added to a 250 mL clear glass vial (Type II), followed by addition of compound (I) (obtained using the procedure of Example 1) at 100 mg/mL. To avoid aggregation, immediately after compounding (i.e. the addition of compound (I) to the solution) a pre-suspension was made by stirring using a magnetic stirrer for 30 minutes. Finally, the milling beads were added. Ytrium stabilized zirconium oxide beads with a diameter of 0.5 mm were added. The vial was closed and placed on a roller mill for the wet milling process. The vial speed was set at 160 rpm. After 64 hr of wet milling a sample was taken for analysis of particle size distribution using laser diffraction to confirm that at least 90% of the suspended particles were inferior to 500 nm in diameter and the milling process was stopped. The white homogeneous nanosuspension was then harvested from the milling beads using a syringe with a 23G needle. After preparation the nanosuspension was stored at 2-8° C.

### Example 3A

### Preparation of Nanosuspension of the Invention by Wet Milling

A nanosuspension of compound (I) was prepared by wet milling following the same procedure as in Example 3 while increasing the concentration of Kollidon 12PF® to 75 mg/g and the concentration of compound (I) to 150 mg/g. The wet milling time was reduced to 28 h and a sample taken for analysis of particle size distribution using laser diffraction confirmed that at least 90% of the suspended particles were inferior to 500 nm in diameter. The particle size distribution Dv (0.10/0.50/0.90/0.99) in μm was found as follows: 0.062/0.120/0.233/0.41.

### Example 3B

### Preparation of Nanosuspension of the Invention by Wet Milling

A nanosuspension of compound (I) was prepared by wet milling following the same procedure as in Example 3 while increasing the concentration of Kollidon 12PF® to 100 mg/g and the concentration of compound (I) to 200 mg/g. The Ytrium stabilized zirconium oxide bead diameter size was increased to 1 mm, the vial speed was set at 120 rpm and the wet milling time was reduced to 40 h. A sample taken for analysis of particle size distribution using laser diffraction confirmed that at least 90% of the suspended particles were inferior to 500 nm in diameter. The particle size distribution Dv (0.10/0.50/0.90/0.99) in μm was found as follows: 0.068/0.140/0.310/0.72.

### Example 3C

### Preparation of Nanosuspension of the Invention by Wet Milling

A nanosuspension of compound (I) was prepared by wet milling following the same procedure as in example 3 while increasing the concentration of Kollidon 12PF® to 150 mg/g and the concentration of compound (I) to 300 mg/g. The Ytrium stabilized zirconium oxide bead diameter size was increased to 1 mm, the vial speed was set at 120 rpm and the wet milling time was reduced to 46 h. A sample taken for analysis of particle size distribution using laser diffraction confirmed that at least 90% of the suspended particles were inferior to 500 nm in diameter. The particle size distribution Dv (0.10/0.50/0.90/0.99) in μm was found as follows: 0.078/0.181/0.492/1.01.

### Example 4

### Nanosuspension (Wet Milling) Particle Size Distribution Analysis

Three different batches of nanosuspension prepared according to the procedure in Example 3 were analysed by laser diffraction analysis to determine the particle size distribution, according to the procedure set out in General Procedures. The samples were also analysed by HPLC to determine the amount of compound (I) in the nanosuspension, also using the procedure set out in General Procedures. The results are summarised in Table 3 and illustrate the reproducibility of the procedure as a very similar particle size distribution was observed in each case.

TABLE 3
Particle size distribution at T = 0 of
nanosuspension formed by wet milling

| | Com- | Dv(0.10)/ | Com- | Kollidon | | |
|---|---|---|---|---|---|---|
| Batch | pound (I) | Dv(0.50)/ | pound (I) | 12PF | | Osmolality |
| No. | (mg/mL) | Dv(0.90) (μm) | (% w/v) | (% w/v) | pH | (mOsm/kg) |
| 1 | 91.6* | 0.066/0.133/ | 9.16 | 4.58 | 4.9 | 45 |
| | | 0.314 | | | | |
| 2 | 93.1* | 0.066/0.135/ | 9.31 | 4.66 | 4.8 | 46 |
| | | 0.358 | | | | |
| 3 | 94.2* | 0.067/0.134/ | 9.42 | 4.71 | 4.7 | 126 |
| | | 0.315 | | | | |

*The theoretical concentration of compound (I) (based on the amount of compound (I) as starting material) was 100 mg/mL

### Example 5

### Nanosuspension (Wet Milling) Stability Analysis

Samples of the nanosuspension prepared according to Example 3 were stored at different temperatures and relative humidities for different lengths of time in order to assess the storage stability of the nanosuspension. Storage stability was assessed in terms of changing particle size distribution, osmolality and pH. The results are summarised in Table 4 and show that the nanosuspension exhibited good stability under typical storage conditions (5° C. and 25° C.).

TABLE 4
Storage stability of nanosuspension formed by wet milling

| Storage | | | | | | | |
|---|---|---|---|---|---|---|---|
| temperature | Test | | | | | | |
| & humidity | interval | Compound | Dv 0.10 | Dv 0.50 | Dv 0.90 | Osmolality | |
| (° C./RH) | (months) | (I) (mg/mL) | (μm) | (μm) | (μm) | (mOsm/kg) | pH |
| T0 | 0 | 92.7 | 0.066 | 0.135 | 0.364 | 46 | 5.0 |
| 5 | 1 | 94.2 | 0.067 | 0.135 | 0.320 | 45 | 4.6 |
| 5 | 2 | 93.1 | 0.067 | 0.138 | 0.487 | 45 | 4.5 |
| 5 | 3 | 93.3 | 0.067 | 0.138 | 0.442 | 45 | 4.6 |
| 5 | 6 | 93.5 | 0.067 | 0.136 | 0.458 | 47 | 4.5 |
| 5 | 9 | 92.2 | 0.067 | 0.140 | 0.854 | 47 | 4.5 |
| 5 | 12 | 92.5 | 0.067 | 0.136 | 0.418 | 45 | 4.5 |
| 25/60% | 1 | 93.8 | 0.069 | 0.145 | 0.446 | 46 | 4.5 |
| 25/60% | 2 | 93.2 | 0.069 | 0.146 | 0.485 | 45 | 4.5 |
| 25/60% | 3 | 93.5 | 0.069 | 0.143 | 0.383 | 45 | 4.5 |
| 25/60% | 6 | 93.1 | 0.069 | 0.145 | 0.621 | 47 | 4.5 |
| 25/60% | 9 | 92.5 | 0.070 | 0.150 | 0.896 | 48 | 4.5 |
| 25/60% | 12 | 92.5 | 0.071 | 0.155 | 0.884 | 46 | 4.5 |

### Example 5A

### Nanosuspension (Wet Milling) Stability Analysis

Samples of the nanosuspension prepared according to Example 3B were stored at different temperatures and relative humidities for different lengths of time in order to assess the storage stability of the nanosuspension. Storage stability was assessed in terms of changing particle size distribution, osmolality and pH. The results are summarised in Table 5 and show that the nanosuspension exhibited good stability under typical storage conditions (5° C. and 25° C.).

TABLE 5
Storage stability of nanosuspension formed by wet milling

| Storage | | | | | | | |
|---|---|---|---|---|---|---|---|
| temperature | Test | Dv | Dv | Dv | Dv | | |
| & humidity | interval | 0.10 | 0.50 | 0.90 | 0.99 | Osmolality | |
| (° C./RH) | (weeks) | (μm) | (μm) | (μm) | (μm) | (mOsm/kg) | pH |
| TO | 0 | 0.068 | 0.140 | 0.310 | 0.72 | 169 | 3.78 |
| 5 | 2 | 0.068 | 0.140 | 0.311 | 0.72 | — | 3.8 |
| 5 | 4 | 0.069 | 0.141 | 0.317 | 0.77 | — | 3.79 |
| 25/60% | 2 | 0.068 | 0.139 | 0.314 | 0.78 | — | 3.79 |
| 25/60% | 4 | 0.070 | 0.145 | 0.336 | 0.85 | — | 3.8 |

### Example 5B

### Nanosuspension (Wet Milling) Stability Analysis

Samples of the nanosuspension prepared according to Example 3C were stored at different temperatures and relative humidities for different lengths of time in order to assess the storage stability of the nanosuspension. Storage stability was assessed in terms of changing particle size distribution, osmolality and pH. The results are summarised in Table 6 and show that the nanosuspension exhibited good stability under typical storage conditions (5° C. and 25° C.).

TABLE 6
Storage stability of nanosuspension formed by wet milling

| Storage | | | | | | | |
|---|---|---|---|---|---|---|---|
| temperature | Test | Dv | Dv | Dv | Dv | | |
| & humidity | interval | 0.10 | 0.50 | 0.90 | 0.99 | Osmolality | |
| (° C./RH) | (weeks) | (μm) | (μm) | (μm) | (μm) | (mOsm/kg) | pH |
| T0 | 0 | 0.078 | 0.181 | 0.492 | 1.01 | 514 | 3.72 |
| 5 | 2 | 0.077 | 0.178 | 0.481 | 0.97 | — | 3.68 |
| 5 | 4 | 0.078 | 0.181 | 0.496 | 1.04 | — | 3.73 |
| 25/60% | 2 | 0.080 | 0.189 | 0.518 | 1.08 | — | 3.66 |
| 25/60% | 4 | 0.080 | 0.188 | 0.534 | 1.14 | — | 3.73 |

### Example 6

### Preparation of Isotonic Nanosuspension (Wet Milling) Formulation for IV Administration

The concentrated nanosuspensions of the invention can be further formulated for an ex-tempore use in a pharmaceutical composition. The following methodology provides examples of isotonic formulations suitable for intravenous (IV) administration.

In support of ex-tempore dilution for IV administration in in vivo tests, dilution of the nanosuspension produced by wet milling (Example 3) in aqueous solutions with osmotic agents was evaluated.

Nanosuspension with a theoretical compound (I) concentration of 100 mg/ml and with an osmolarity of 46 mOsm/kg and a density of 1.031 g/mL was diluted in a ratio 1/3, 1/1 or 3/1 v/v using an aqueous solution of glucose, mannitol or sodium chloride. In a second series, nanosuspension with a theoretical compound (I) concentration of 100 mg/mL was diluted in a ratio of 1/1, 3/1, 6/1, 10/1 and 25/1 using an aqueous solution of sodium chloride. The concentration of the solution was adapted as a function of osmolarity. An osmolarity of the diluted nanosuspension of 280-320 mOsm/L was targeted.

Stock solutions of mannitol, glucose and sodium chloride were prepared in different concentrations in purified water. Dilutions of the nanosuspension in aqueous solutions of mannitol, glucose and sodium chloride were prepared by transferring a specified volume of nanosuspension to a glass beaker. A specified volume of stock solution was then added and the suspension was stirred using a magnetic stirrer. A final volume of 25 mL was prepared for each combination, resulting in white homogeneous suspensions. The concentrations of mannitol, glucose and sodium chloride used in the diluting solutions, the dilution ratio and the analytical data of these resulting novel nanosuspension formulations are provided in Table 7 below. The osmolarity results all fall within the range 270-330 mOsm/kg recommended by the European Pharmacopeia for a parenteral isotonic solution/suspension.

TABLE 7
Nanosuspension formulations suitable for intravenous administration.

| | | Excipient | | | Osmolarity | Osmolarity | | PSD^{d} |
|---|---|---|---|---|---|---|---|---|
| | | concentration* | Dilution | Density | 1 | 2 | | Dv(0.10)/Dv(0.50)/Dv(0.90) |
| No. | Excipient | (mg/mL) | ratio^{a} | (g/mL) | (mOsm/L)^{b} | (mOsm/L)^{c} | pH | μm |
| 1 | NaCl | 11.23 | 1/3 | 1.012 | 280 | 293 | 6.0 | 0.067/0.137/0.327 |
| 2 | NaCl | 30.91 | 3/1 | 1.029 | 279 | 317 | 4.8 | 0.067/0.137/0.366 |
| 3 | D- | 65.07 | 1/3 | 1.023 | 298 | 312 | 5.2 | 0.064/0.126/0.266 |
| | mannitol | | | | | | | |
| 4 | D- | 155.44 | 3/1 | 1.037 | 283 | 302 | 5.0 | 0.067/0.135/0.333 |
| | mannitol | | | | | | | |
| 5 | D-(+)- | 63.81 | 1/3 | 1.020 | 298 | 312 | 6.4 | 0.066/0.134/0.323 |
| | glucose | | | | | | | |
| 6 | D-(+)- | 150.02 | 3/1 | 1.038 | 279 | 299 | 5.6 | 0.065/0.130/0.303 |
| | glucose | | | | | | | |
| 7 | NaCl | 16.17 | 1/1 | 1.021 | 279 | 303 | 6.2 | 0.073/0.140/0.344 |
| 8 | NaCl | 16.17 | 1/1 | ND | ND | ND | ND | ND |
| 9 | NaCl | 30.96 | 3/1 | ND | ND | ND | ND | ND |
| 10 | NaCl | 53.16 | 6/1 | ND | ND | ND | ND | ND |
| 11 | NaCl | 82.76 | 10/1 | ND | ND | ND | ND | ND |
| 12 | NaCl | 193.77 | 25/1 | ND | ND | ND | ND | ND |

*concentration of excipient stock solution
^{a}(volume of nanosuspension/volume excipient)
^{b}weighted average of osmolarity of components
^{c}calculated from osmolality and density
^{d}particle size distribution

### Example 6A

### Preparation of Isotonic Nanosuspension (Wet Milling) Formulation for IV Administration

An aqueous solution of sodium chloride was added to a nanosuspension of 200 mg/g of compound (I) prepared according to Example 3B. Then, the diluted nanosuspension was homogenized by manually shaking. The theoretical concentration of this isotonic nanosuspension is 100 mg/mL of compound (I), 50 mg/mL of Kollidon 12PF® and 6.9 mg/mL of NaCl.

This nanosuspension demonstrated an acceptable assay of the compound (I) of 99.3 mg/mL and a suitable osmolality of 309 mOsm/kg for IV administration. A sample taken for analysis of particle size distribution using laser diffraction confirmed that at least 90% of the suspended particles were inferior to 500 nm in diameter. The particle size distribution Dv (0.10/0.50/0.90) in μm was found as follows: 0.068/0.137/0.293.

### Example 6B

### Preparation of Isotonic Nanosuspension (Wet Milling) Formulation for IV Administration

An aqueous solution of glucose was added to a nanosuspension of 200 mg/g of compound (I) prepared according to Example 3B. Then, the diluted nanosuspension was homogenized by manually shaking. The theoretical concentration of this isotonic nanosuspension is 100 mg/mL of compound (I), 50 mg/mL of Kollidon 12PF® and 34.6 mg/mL of glucose.

This nanosuspension demonstrated an acceptable assay of the compound (I) of 99.3 mg/mL and a suitable osmolality of 294 mOsm/kg for IV administration. A sample taken for analysis of particle size distribution using laser diffraction confirmed that at least 90% of the suspended particles were inferior to 500 nm in diameter. The particle size distribution Dv (0.10/0.50/0.90) in μm was found as follows: 0.065/0.129/0.268.

### Example 7

### Stability of Isotonic Nanosuspension (Wet Milling) Formulation for IV Administration

The short-term stability of the isotonic nanosuspensions of Example 6 in closed vials was assessed. Storage stability was assessed in terms of changing particle size distribution, osmolarity and pH. The results are summarised in Table 8 and demonstrate that these novel formulations are suitable for an ex-tempore IV administration since the osmolality, the active principle concentration, the pH and the particle size distribution are relatively stable over 48-69 h.

TABLE 8
Short-term stability of isotonic nanosuspension

| | Storage | | | | Com- | | | |
|---|---|---|---|---|---|---|---|---|
| | temper- | | Os- | | pound | | | |
| | ature & | Time | molality | | (I) | Dv | Dv | Dv |
| | humidity | point | (mOsm/ | | (mg/ | 0.10 | 0.50 | 0.90 |
| No. | (° C./RH) | (h) | kg) | pH | mL) | (μm) | (μm) | (μm) |
| 1 | — | 0 | 289 | 6.02 | 23.4 | 0.067 | 0.137 | 0.327 |
| 1 | 5 | 24 | 289 | 5.17 | 23.7 | 0.065 | 0.130 | 0.290 |
| 1 | 5 | 48 | 289 | 5.31 | 23.7 | 0.066 | 0.134 | 0.303 |
| 1 | 5 | 69 | — | — | — | 0.069 | 0.146 | 0.562 |
| 1 | 25/60% | 24 | 289 | 5.13 | 23.8 | 0.068 | 0.140 | 0.389 |
| 1 | 25/60% | 48 | 289 | 5.45 | 23.6 | 0.066 | 0.135 | 0.365 |
| 1 | 25/60% | 69 | — | — | — | 0.069 | 0.146 | 0.551 |
| 2 | — | 0 | 308 | 4.84 | 71.2 | 0.067 | 0.137 | 0.366 |
| 2 | 5 | 24 | 309 | 4.66 | 70.9 | 0.064 | 0.128 | 0.277 |
| 2 | 5 | 48 | 310 | 4.73 | 71.2 | — | — | — |
| 2 | 5 | 69 | — | — | — | 0.068 | 0.144 | 0.563 |
| 2 | 25/60% | 24 | 309 | 4.69 | 71.4 | — | — | — |
| 2 | 25/60% | 48 | 309 | 4.61 | 71.2 | — | — | — |
| 2 | 25/60% | 69 | — | — | — | 0.069 | 0.143 | 0.386 |
| 3 | — | 0 | 305 | 5.22 | 23.7 | 0.064 | 0.126 | 0.266 |
| 3 | 5 | 24 | 305 | 5.07 | 23.8 | 0.068 | 0.139 | 0.364 |
| 3 | 5 | 48 | 306 | 5.35 | 23.8 | — | — | — |
| 3 | 5 | 69 | — | — | — | 0.066 | 0.134 | 0.359 |
| 3 | 25/60% | 24 | 306 | 5.13 | 23.8 | 0.069 | 0.146 | 0.594 |
| 3 | 25/60% | 48 | 306 | 5.37 | 23.8 | — | — | — |
| 3 | 25/60% | 69 | — | — | — | 0.067 | 0.137 | 0.380 |
| 4 | — | 0 | 291 | 4.97 | 71.2 | 0.067 | 0.135 | 0.333 |
| 4 | 5 | 24 | 292 | 4.72 | 71.4 | — | — | — |
| 4 | 5 | 48 | 292 | 4.73 | 71.3 | — | — | — |
| 4 | 5 | 69 | — | — | — | 0.067 | 0.139 | 0.426 |
| 4 | 25/60% | 24 | 292 | 4.80 | 71.7 | — | — | — |
| 4 | 25/60% | 48 | 293 | 4.77 | 70.7 | — | — | — |
| 4 | 25/60% | 69 | — | — | — | 0.068 | 0.140 | 0.399 |
| 5 | — | 0 | 306 | 6.35 | 23.2 | 0.066 | 0.134 | 0.324 |
| 5 | 5 | 24 | 306 | 5.27 | 23.6 | — | — | — |
| 5 | 5 | 48 | 307 | 5.26 | 23.6 | — | — | — |
| 5 | 5 | 69 | — | — | — | 0.067 | 0.139 | 0.411 |
| 5 | 25/60% | 24 | 307 | 5.13 | 23.6 | — | — | — |
| 5 | 25/60% | 48 | 307 | 5.46 | 23.7 | — | — | — |
| 5 | 25/60% | 69 | — | — | — | 0.067 | 0.137 | 0.383 |
| 6 | — | 0 | 288 | 5.56 | 70.7 | 0.065 | 0.130 | 0.303 |
| 6 | 5 | 24 | 285 | 4.82 | 70.7 | — | — | — |
| 6 | 5 | 48 | 287 | 4.81 | 71.2 | — | — | — |
| 6 | 5 | 69 | — | — | — | 0.065 | 0.131 | 0.320 |
| 6 | 25/60% | 24 | 288 | 4.70 | 71.2 | — | — | — |
| 6 | 25/60% | 48 | 290 | 4.72 | 71.1 | — | — | — |
| 6 | 25/60% | 69 | — | — | — | 0.068 | 0.140 | 0.401 |
| 7 | — | 0 | 297 | 6.2 | — | 0.073 | 0.140 | 0.344 |
| 7 | 5 | 48 | 299 | 5.7 | — | 0.065 | 0.133 | 0.343 |

### Example 7A

### Stability of Isotonic Nanosuspension (Wet Milling) Formulation for IV Administration

Samples of the nanosuspension prepared according to Example 6A were stored in closed vials at different temperatures and relative humidities for different lengths of time in order to assess the storage stability of the nanosuspension. Storage stability was assessed in terms of changing particle size distribution, osmolality and pH. The results are summarised in Table 9 and show that the nanosuspension exhibited good stability under typical storage conditions (5° C. and 25° C.) and intermediate stability under accelerated storage conditions (40° C. with 75% RH).

TABLE 9
Storage stability of nanosuspension formed by wet milling

| Storage | | | | | | |
|---|---|---|---|---|---|---|
| temperature | Test | | | | | |
| & humidity | interval | Dv 0.10 | Dv 0.50 | Dv 0.90 | Osmolality | |
| (° C./RH) | (weeks) | (μm) | (μm) | (μm) | (mOsm/kg) | pH |
| T0 | 0 | 0.068 | 0.137 | 0.293 | 309 | 3.77 |
| 5 | 2 | 0.068 | 0.140 | 0.318 | — | 4.00 |
| 5 | 6 | 0.067 | 0.136 | 0.308 | — | 3.92 |
| 25/60% | 2 | 0.071 | 0.152 | 0.414 | — | 3.98 |
| 25/60% | 6 | 0.071 | 0.155 | 0.438 | — | 3.85 |
| 40/75% | 2 | 0.077 | 0.187 | 0.774 | — | 3.91 |
| 40/75% | 6 | 0.078 | 0.184 | 0.587 | — | 3.79 |

### Example 7B

### Stability of Isotonic Nanosuspension (Wet Milling) Formulation for IV Administration

Samples of the nanosuspension prepared according to Example 6B were stored in closed vials at different temperatures and relative humidities for different lengths of time in order to assess the storage stability of the nanosuspension. Storage stability was assessed in terms of changing particle size distribution, osmolality and pH. The results are summarised in Table 10 and show that the nanosuspension exhibited good stability under typical storage conditions (5° C. and 25° C.) but also under accelerated storage conditions (40° C. with 75% RH).

TABLE 10
Storage stability of nanosuspension formed by wet milling

| Storage | | | | | | |
|---|---|---|---|---|---|---|
| temperature | Test | | | | | |
| & humidity | interval | Dv 0.10 | Dv 0.50 | Dv 0.90 | Osmolality | |
| (° C./RH) | (weeks) | (μm) | (μm) | (μm) | (mOsm/kg) | pH |
| T0 | 0 | 0.065 | 0.129 | 0.268 | 294 | 3.84 |
| 5 | 2 | 0.065 | 0.129 | 0.266 | — | 4.04 |
| 5 | 6 | 0.065 | 0.129 | 0.268 | — | 3.98 |
| 25/60% | 2 | 0.065 | 0.131 | 0.272 | — | 4.02 |
| 25/60% | 6 | 0.065 | 0.128 | 0.266 | — | 3.91 |
| 40/75% | 2 | 0.068 | 0.139 | 0.301 | — | 4.01 |
| 40/75% | 6 | 0.069 | 0.141 | 0.311 | — | 3.90 |

The results of Examples 7, 7A and 7B show that methods of the invention are suitable for the preparation of isotonic nanosuspensions of compound (I) suitable for IV administration that are stable for at least 6 weeks.

### Example 8

### Preparation of Nanosuspension of the Invention by Controlled Precipitation

A nanosuspension of compound (I) was prepared by controlled precipitation as follows. 5.33 g of compound (I) (obtained using the procedure of Example 2) was dissolved in 74 mL of ethyl acetate. The solution was then combined with 400 mL of 40% w/w Kollidon 12 in deionized water. The mixture was heated and stirred with a magnetic stir bar for 4 h at 40° C., then left stirring uncovered overnight at room temperature. A 1 L jacketed beaker was filled with 928 mL deionized water at 2° C. While stirring at 600 rpm, 32 mL of the compound (I)/PVP solution was pumped into the beaker at a rate of 10 mL/min using a 50 mL syringe and a 19 gauge needle submerged under the surface of water. The solution immediately turned hazy and was left stirring overnight at 2° C., and after 16 hrs turned off cooling and left to warm up slowly. This process was repeated three additional times in separate batches. The solutions were washed and concentrated using the cross flow system PureTec Autopurification System with Sartocon Hydrostart 30 kD filter and size 15 tubing. After priming the filter, 460 mL of suspension was filtered in a loop at 180 rpm for 5 hours. This process was used to wash the suspension with 1.5 L of deionized water and to reduce the final volume to 40 mL. The concentrated solutions from each of the four batches were combined, concentrated further to 40 mL, and centrifuged at 400 G for 20 minutes. To remove the residual Kollidon 12, the final suspension was washed 20 times with a total of 800 mL to produce the final 40 mL sample.

### Example 9

### Nanosuspension (Controlled Precipitation) Particle Size Distribution Analysis

A particle size analysis of the nanosuspension prepared according to Example 8 was carried out as set out in the General Procedures and the results are summarized in FIG. 2 and Table 11 below, from which is can be seen that the protocol set out in Example 8 produces a nanosususpension with a mean particle size of 391.5 nm (intensity %) with few microparticles (i.e. particles >1 μm).

TABLE 11
Particle size distribution summary

| | Parameter | Calculated Results | | | | | |
|---|---|---|---|---|---|---|---|
| | SDP Range | Size | % amt | Std. Dev | | | |
| Angle | (nm) | (nm) | (nm) | (nm) | Mean Size (nm) | Mean SD (nm) | % Dust* |
| 90.0° | 3.0-3000.0 | 391.5 | 100.00 | 168.6 | 391.5 | 168.6 | 1.967 |

*particles with mean size > 1 μm

The data was then manipulated via curve fitting to provide approximate values for the volume distribution, as follows:

Dv(0.10): 200.11 nm (calculated with 4th order polynomial fit)
Dv(0.50): 392.8 nm (calculated with 4th order polynomial fit)
Dv(0.90): 534.168 nm (calculated with linear fit between two data points).

### Example 10

### Nanosuspension (Controlled Precipitation) Thermal Analysis

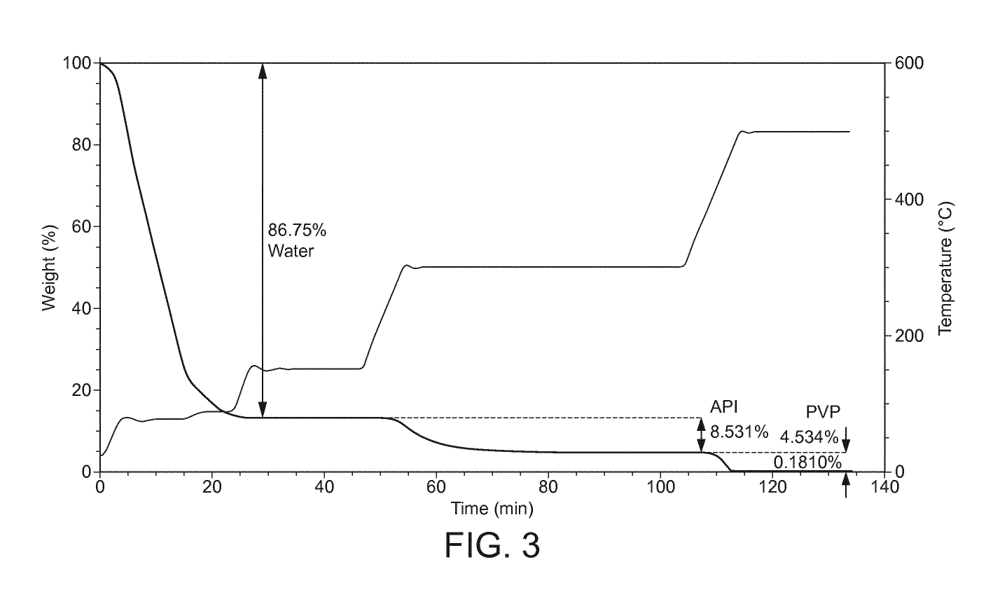

Thermogravimetric analysis (TA Instruments Q600) was used to determine the final solids content of the nanosuspension prepared in Example 8. The sample was heated to 75° C. for 20 min, 150° C. for 20 min, 150° C. for 60 minutes, then 500° C. for 30 minutes. The ramp rate was 20° C./min. Three noticeable weight losses were observed that coincide with water, compound (I) and PVP 12, respectively. It was presumed that a majority of the residual mass after 500° C. was compound (I), and therefore included in the final concentration calculation of 8.80%. The results are shown in FIG. 3 (which also shows a DSC analysis of the nanosuspension) which indicates a content of compound (I) of 88 mg/mL (assuming density of 1 g/mL) with 4.66% w/w of Kollidon 12, which is within acceptable tolerances. The amount of PVP in the nanosuspension may be further decreased by diluting to a final isotonic pharmaceutical formulation, as described in Example 9.

### Example 11

### Nanosuspension (Controlled Precipitation) Zeta Potential Analysis

The zeta potential of the nanosuspension prepared according to Example 8 was measured following the procedure set out in the General Procedures. A mean zeta potential of −43.41 mV was observed, indicating good potential stability of this nanosuspension.

### Example 12

### Stability of Nanosuspension (Wet Milling) to Gamma-Irradiation

Samples of the nanosuspension of 100 mg/mL of compound (I) prepared by dilution of a 200 mg/g initial nanosuspension prepared according to Example 3B with water to reach a final concentration of 100 mg/mL of compound (I), were subjected to increased doses of gamma rays up to 40 kGy. The samples were filled in 6 mL clear glass vials that were packed in a cardboard box. The dose of gamma rays applied was calculated based on measured values from dosimeters attached to the box. The results are summarised in Table 12 and show that the nanosuspension exhibited good stability toward this sterilization treatment.

TABLE 12
Gamma-irradiation stability of nanosuspension
formed by wet milling

| Irradiation | Dv 0.10 | Dv 0.50 | Dv 0.90 | Osmolality | |
|---|---|---|---|---|---|
| conditions | (μm) | (μm) | (μm) | (mOsm/kg) | pH |
| Before | 0.069 | 0.143 | 0.325 | 52.5 | 4.08 |
| irradiation | | | | | |
| 25 kGy | 0.069 | 0.144 | 0.329 | 50 | 5.58 |
| 40 kGy | 0.069 | 0.144 | 0.328 | 51.5 | 5.38 |

### Example 13

### Stability of Buffered Isotonic Nanosuspension (Wet Milling) to Gamma-Irradiation

Samples of the buffered isotonic nanosuspension of 100 mg/mL of compound (I) prepared by dilution of a 200 mg/g initial nanosuspension prepared according to Example 3B with an acetic acid buffer (pH 4.3) and NaCl to reach a final concentration of 100 mg/mL of compound (I), were subjected to increased doses of gamma rays up to 40 kGy. The samples were filled in 6 mL clear glass vials that were packed in a cardboard box. The dose of gamma rays applied was calculated based on measured values from dosimeters attached to the box. The results are summarised in Table 13 and show that the nanosuspension exhibited good stability toward this sterilization treatment.

TABLE 13
Gamma-irradiation stability of buffered isotonic
nanosuspension formed by wet milling

| Irradiation | Dv 0.10 | Dv 0.50 | Dv 0.90 | Osmolality | |
|---|---|---|---|---|---|
| conditions | (μm) | (μm) | (μm) | (mOsm/kg) | pH |
| Before | 0.072 | 0.158 | 0.517 | 283 | 4.18 |
| irradiation | | | | | |
| 25 kGy | 0.075 | 0.171 | 0.661 | 283 | 4.20 |
| 40 kGy | 0.073 | 0.165 | 0.588 | 276.5 | 4.21 |

### Example 14

### Time Stability of Gamma-Irradiated Isotonic Nanosuspension (Wet Milling)

Samples of the isotonic NaCl nanosuspension of compound (I) prepared according to Example 6A were irradiated at 47.8-53.8 kGy. The samples were filled in 6 mL clear glass vials that were packed in a cardboard box. The minimum and maximum dose of gamma rays applied was calculated based on measured values from dosimeters attached to the box. The results are summarised in Table 14 and show that the nanosuspension exhibited moderate stability toward this sterilization treatment and exhibited good stability under typical storage conditions (5° C. and 25° C.) but also under accelerated storage conditions (40° C. with 75% RH).

TABLE 14
Gamma-irradiation stability of NaCl isotonic nanosuspension
of compound (I) formed by wet milling

| Storage | Test | Dv | Dv | Dv | | |
|---|---|---|---|---|---|---|
| temperature & | interval | 0.10 | 0.50 | 0.90 | Osmolality | |
| humidity (° C./RH) | (weeks) | (μm) | (μm) | (μm) | (mOsm/kg) | pH |
| No irradiation | | 0.069 | 0.144 | 0.372 | 312 | 3.75 |
| Immediately after | 0 | 0.074 | 0.167 | 0.662 | 308 | 5.79 |
| irradiation-T0 | | | | | | |
| 5 | 2 | 0.073 | 0.162 | 0.581 | 309 | 5.87 |
| 25/60% | 2 | 0.073 | 0.163 | 0.585 | 305 | 5.85 |
| 40/75% | 2 | 0.074 | 0.166 | 0.599 | 308 | 5.69 |

### Example 14A

### Time Stability of Gamma-Irradiated Isotonic Nanosuspension (Wet Milling)

Samples of the isotonic glucose nanosuspension of compound (I) prepared according to Example 6B were irradiated at 47.8-53.8 kGy. The samples were filled in 6 mL clear glass vials that were packed in a cardboard box. The minimum and maximum dose of gamma rays applied was calculated based on measured values from dosimeters attached to the box. The results are summarised in Table 15 and show that the nanosuspension exhibited good stability toward this sterilization treatment and exhibited good stability under typical storage conditions (5° C. and 25° C.) but also under accelerated storage conditions (40° C. with 75% RH).

TABLE 15
Gamma-irradiation stability of glucose isotonic nanosuspension
of compound (I) formed by wet milling

| Storage | Test | | | | | |
|---|---|---|---|---|---|---|
| temperature & | interval | Dv 0.10 | Dv 0.50 | Dv 0.90 | Osmolality | |
| humidity (° C./RH) | (weeks) | (μm) | (μm) | (μm) | (mOsm/kg) | pH |
| No irradiation | | 0.067 | 0.134 | 0.281 | 294 | 3.82 |
| Immediately after | 0 | 0.068 | 0.137 | 0.303 | 291 | 3.71 |
| irradiation-T0 | | | | | | |
| 5 | 2 | 0.068 | 0.137 | 0.303 | 289 | 3.69 |
| 25/60% | 2 | 0.067 | 0.137 | 0.303 | 291 | 3.68 |
| 40/75% | 2 | 0.068 | 0.140 | 0.347 | 290 | 3.65 |

Examples 12, 13, 14 and 14A show that methods of the invention are suitable for the preparation of nanosuspensions of compound (I) suitable for IV administration that are stable after gamma-irradiation up to at least 40 kGy.

### Example 15

### Scaled-Up Preparation of Nanosuspension of the Invention by Wet Milling

A nanosuspension of compound (I) was prepared by wet milling following the same procedure as in Example 3 while scaling up the amount of compound (I) to 650 g in a 2 L bottle achieving a final concentration of 200 mg/g of compound (I) and 100 mg/g of Kollidon 12PF®. The wet milling time was 22 h with a vial speed set at 80 rpm and beads diameter of 0.5 mm. A sample taken for analysis of particle size distribution using laser diffraction confirmed that at least 90% of the suspended particles were inferior to 500 nm in diameter. The particle size distribution Dv (0.10/0.50/0.90/0.99) in μm was found as follows: 0.065/0.128/0.264/0.600. This result indicates that such preparations are suitable for scale-up.

### Example 16

### In Vivo Systemic Mouse Infection Model Against S. aureus

The aim of this study was to compare the in vivo efficacy of the prior art historical formulation with a novel nanosuspension of the present invention using a murine systemic infection model against both a susceptible (MSSA) and a methicillin resistant (MRSA) strains of S. aureus.

Methods

Three independent studies were run against each isolate (one MSSA and one MRSA), the data for the three studies combined and analysis performed. Bacterial isolates were grown to log phase in a liquid culture. Bacteria were diluted in 8% mucin to a concentration to achieve at least 90% mortality within 48 hours after infection. Six CD-1 female mice, 18-20 grams, were allocated per compound (I) concentration and were infected with 0.5 ml of bacterial suspension via intraperitoneal injection. Five minutes post-infection, mice were treated via intravenous administration. A nanosuspension of the invention (prepared according to Example 3) was prepared fresh just prior to single IV dose delivery at 50, 100 and 200 mg/kg (dosage of compound (I)). The prior art historical formulation of compound (I) (formulated at 10 mg/mL in 20% HPBCD, and 1% glucose) and vancomycin (Sigma Aldrich) were also administered IV at one dose and were used as comparators. Infection control mice were dosed with saline. Survival was assessed for 48 hours post infection. Date of death was recorded and a Probit analysis was performed to determine the PD₅₀ (50% protective dose).

Results

The results are summarised in Tables 16 and 17 below. A nanosuspension of the invention dosed at 90 mg/mL with 5% PVP 12PF as stabilising agent demonstrated significant activity with an estimated combined (n=3) PD₅₀ value of less than 50 mg/kg against the susceptible isolate MSSA and with an estimated combined (n=3) PD₅₀ value of is 83.4 mg/kg against the methicillin resistant isolate MRSA. In vivo efficacy of the new nanosuspension was in the same range of efficacy as the prior art historical formulation in this murine model of S. aureus systemic infection.

TABLE 16
Combined Results (n = 3) of a Systemic
Mouse Infection Model against MSSA
Systemic Infection Studies MSSA (n = 3)

| | Dose | | % | |
|---|---|---|---|---|
| Test Article | (mg/kg) | Survival | survival | PD₅₀ |
| Nanosuspension of | 200 | 12/14* | 85.7 | <50 mg/kg |
| the invention | 100 | 13/18 | 72.2 | |
| | 50 | 11/18 | 61.1 | |
| Prior art historical | 100 | 15/18 | 83.3 | <100 mg/kg |
| formulation | | | | |
| Vancomycin | 50 | 18/18 | 100 | <50 mg/kg |

*4 animals died over the course of 3 studies for reasons unrelated to the study

TABLE 17
Combined Results (n = 3) of a Systemic
Mouse Infection Model against MRSA
Systemic Infection Studies MRSA 300 (n = 3)

| | Dose | | % | PD₅₀ |
|---|---|---|---|---|
| Test Article | (mg/kg) | Survival | survival | (95% C.I.) |
| Nanosuspension of | 200 | 11/15* | 73.3 | 82.0 mg/kg |
| the invention | 100 | 9/15* | 60 | (−12.9-176.9) |
| | 50 | 6/16* | 37.5 | |
| Prior art historical | 100 | 9/18 | 50 | ~100 mg/kg |
| formulation | | | | |
| Vancomycin | 50 | 18/18 | 100 | <50 mg/kg |

*some animals died for reasons unrelated to the study

Conclusion

A novel nanosuspension of the invention provided significant protection against MSSA and MRSA with comparable survival percentages to the prior art historical formulation of compound (I) in a murine model of systemic infection.

### Example 17

### In Vivo Neutropenic Mouse Thigh Infection Model Against S. aureus

The aim of this study was to compare the in vivo efficacy of the prior art historical formulation with a novel nanosuspension of the present invention using a neutropenic mouse thigh infection model against both a susceptible (MSSA) and a methicillin resistant (MRSA) strain of S. aureus.

Method

Seven independent studies were performed. The S. aureus isolates were prepared from an overnight plate culture and re-suspended to a pre-determined concentration confirmed by Optical Density. The final inoculum concentration was determined through CFU enumeration of the prepared inoculum. For each study, four CD-1 female mice, 18-20 grams were allocated per dose group and were rendered neutropenic by 2 consecutive doses of cyclophosphamide (150 and 100 mg/kg) on days −4 and −1. Mice were injected with 0.1 ml of bacterial suspension via intramuscular injection. Ninety minutes post infection, mice were treated via intravenous administration. A nanosuspension of the invention (prepared according to Example 3) was prepared fresh just prior to dose delivery with mice being dosed at 50 mg/kg, 100 mg/kg and 200 mg/kg of compound (I). The prior art historical formulation and vancomycin were delivered by IV at one dose and used as comparators. Infection control mice were dosed with saline.

Results

Against the susceptible isolate MSSA, the nanosuspension of the invention was as active as the prior art historical formulation of compound (I) with a bioburden reduction of 0.61 log 10 CFU/thigh at 100 mg/kg (n=1). Against the resistant isolate MRSA, the nanosuspension of the invention was as active as the prior art historical formulation of compound (I) with a bioburden reduction of 0.79 log 10 CFU/thigh at 100 mg/kg (n=1).

Conclusion

A novel nanosuspension of the invention provided comparable efficacy against MSSA and MRSA compared with the prior art historical formulation of compound (I) in a neutropenic mouse thigh infection model.

### Example 18

### In Vivo Tissue Distribution Study

A tissue distribution study was conducted in male Sprague Dawley rats to compare the pharmacokinetic (PK) profile in plasma, liver, heart, lungs, skin and bone of compound (I) to the prior art historical formulation. Animals were administered with the prior art historical formulation (F3) or with nanosuspensions of the invention (F1 and F2) at 200 mg of compound (I) per kg as a single 10 minute infusion in the caudal vein.

The three formulations administered were as follows and the results are summarized in Table 18 below:

Formulation #1 (F1): Nanosuspension of the invention prepared substantially according to Example 3, containing of 96.9 mg of compound (I)/mL in 5% PVP 12PF.
Formulation #2 (F2): Nanosuspension of the invention prepared substantially according to Example 8, containing 88 mg of compound (I)/mL in 5% PVP K12.

Prior art historical formulation (F3): Cyclodextrin-based complex which is a concentrate containing 10.28 mg of compound (I)/mL in 20% HPBCD and 1% glucose.

TABLE 18
Mean rat pharmacokinetic parameters of compound (I) following single 10 minute
intravenous infusion of compound (I) formulations (10 mg/mL) at 200 mg/kg

| | | AUC₀₋ₗₐₛₜ ± SE | AUC₁₂ | AUC | Cₘₐₓ ± SE | t_{1/2} |
|---|---|---|---|---|---|---|
| Matrix | Formulation | (ng*h/mL) | (ng*h/mL) | (ng*h/mL) | (ng/mL) | (h) |
| Bones | F1 | 49,061 ± 3,405 | 49,026 | 50,193 | 82,633 ± 7,186 | 5.22 |
| | F2 | 67,574 ± 12,149 | 67,425 | 68,010 | 72,933 ± 14,673 | 2.02 |
| | F3 | 62,855 ± 3,366 | 62,810 | 64,798 | 87,467 ± 7,131 | 6.38 |
| Heart | F1 | 119,684 ± 12,627 | 119,677 | 121,107 | 231,867 ± 46,965 | 22.40 |
| | F2 | 94,122 ± 12,983 | 94,116 | 96,611 | 167,667 ± 53,249 | 47.66 |
| | F3 | 157,198 ± 6,322 | 157,191 | 157,729 | 231,167 ± 14,214 | 10.16 |
| Liver | F1 | 115,397 ± 6,289 | 114,934 | NC | 190,333 ± 21,543 | NC |
| | F2 | 135,138 ± 10,949 | 134,909 | 149,080 | 206,667 ± 23,333 | 8.13 |
| | F3 | 169,159 ± 12,205 | 168,749 | 196,772 | 260,333 ± 29,812 | 8.80 |
| Lung | F1 | 97,685 ± 5,303 | 97,620 | 101,966 | 151,667 ± 9,207 | 8.68 |
| | F2 | 88,962 ± 6,667 | 88,913 | 91,650 | 161,333 ± 17,381 | 7.50 |
| | F3 | 131,788 ± 5,267 | 131,727 | 135,420 | 207,333 ± 17,130 | 8.02 |
| Plasma | F1 | 43,683 ± 2,741 | 43,678 | 44,644 | 59,433 ± 5,659 | 21.6 |
| | F2 | 52,981 ± 1,998 | 52,966 | 55,863 | 73,933 ± 5,685 | 23.4 |
| | F3 | 89,962 ± 3,697 | 89,957 | 90,195 | 141,333 ± 9,333 | 6.46 |
| Skin | F1 | 55,031 ± 4,121 | 55,021 | 55,146 | 90,433 ± 2,672 | 3.19 |
| | F2 | 51,937 ± 7,395 | 52,056 | 51,986 | 83,233 ± 26,889 | 0.57 |
| | F3 | 94,469 ± 5,851 | 94,458 | 94,583 | 149,000 ± 12,014 | 3.10 |

A higher exposure in plasma and tissues was observed for the prior art historical formulation compared with the pharmaceutical aqueous nanosuspensions of the invention, as the plasma clearance of the historical formulation was halved compared with the pharmaceutical aqueous nanosuspensions of the invention, resulting in greater plasma exposure. Formulations F1 (prepared by wet milling—Example 3) and F2 (prepared by controlled precipitation—Example 8) were observed to be roughly equivalent in terms of plasma and tissue exposure.

However, despite the nanosuspensions of the invention having a lower plasma concentration that the prior art historical formulation, the overall tissue-plasma partition coefficient was higher for the pharmaceutical aqueous nanosuspensions of the invention in all matrices when compared with the prior art historical formulation, as can be seen from Table 19 below. Formulations 1 and 2 were found to be roughly equivalent in this regard.

TABLE 19
Rat tissue-plasma partition coefficients for the three formulations

| Matrix | Formulation | T/P(AUC₀-ₗₐₛₜ) | T/P(AUC₁₂) | T/P(Cₘₐₓ) |
|---|---|---|---|---|
| Bones | F1 | 1.12 | 1.12 | 1.39 |
| | F2 | 1.28 | 1.27 | 0.99 |
| | F3 | 0.70 | 0.70 | 0.62 |
| Heart | F1 | 2.74 | 2.74 | 3.90 |
| | F2 | 1.78 | 1.78 | 2.27 |
| | F3 | 1.75 | 1.75 | 1.64 |
| Liver | F1 | 2.64 | 2.63 | 3.20 |
| | F2 | 2.55 | 2.55 | 2.80 |
| | F3 | 1.88 | 1.88 | 1.84 |
| Lung | F1 | 2.24 | 2.23 | 2.55 |
| | F2 | 1.68 | 1.68 | 2.18 |
| | F3 | 1.46 | 1.46 | 1.47 |
| Skin | F1 | 1.26 | 1.26 | 1.52 |
| | F2 | 0.98 | 0.98 | 1.13 |
| | F3 | 1.05 | 1.05 | 1.05 |

This implies that the pharmaceutical aqueous nanosuspensions of the invention provide better penetration into the tissues than the prior art historical formulation, thereby allowing a lower concentration of formulation to be administered.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

1. A pharmaceutical aqueous nanosuspension comprising 4-(4-ethyl-5-fluoro-2-hydroxyphenoxy)-3-fluorobenzamide in nanoparticulate form and a stabilizing agent.

2. A pharmaceutical aqueous nanosuspension according to claim 1, wherein the stabilizing agent is a surfactant or a surfactant polymer.

3. A pharmaceutical aqueous nanosuspension according to claim 2, wherein the stabilizing agent is polyvinylpyrrolidone.

4. A pharmaceutical aqueous nanosuspension according to claim 1, which is substantially free of cyclodextrin.

5. A pharmaceutical aqueous nanosuspension according to claim 1, wherein the volume median diameter (Dv(0.50)) of the nanosuspension is between about 0.050 μm and about 0.600 μm.

6. A pharmaceutical aqueous nanosuspension according to claim 1, wherein the concentration of 4-(4-ethyl-5-fluoro-2-hydroxyphenoxy)-3-fluorobenzamide in the nanosuspension is at least 20 mg/mL, for example at least 30 mg/mL, at least 40 mg/mL, at least 50 mg/mL, at least 60 mg/mL, at least 70 mg/mL or at least 80 mg/mL.

7. A pharmaceutical aqueous nanosuspension according to claim 1, additionally comprising a tonicity modifier such as NaCl, mannitol or glucose.

8. (canceled)

9. A pharmaceutical aqueous nanosuspension according to claim 1, which has osmolality of between about 250 mOsm/kg and about 350 mOsm/kg, for example between about 280 mOsm/kg and about 320 mOsm/kg.

10. A pharmaceutical aqueous nanosuspension according to claim 1, wherein the nanosuspension is isotonic.

11. A pharmaceutical aqueous nanosuspension according to claim 1, wherein the nanosuspension is stable to irradiation by gamma rays of up to 40 kGy.

12. A pharmaceutical aqueous nanosuspension according to claim 1, wherein the nanosuspension is stable to storage at 25° C. and 60% RH for up to 6 weeks.

13. A pharmaceutical aqueous nanosuspension according to claim 1, for parenteral administration, for example intravenous administration.

14. (canceled)

15. A pharmaceutical aqueous nanosuspension according to claim 1, further comprising one or more additional medicaments, for example an anti-bacterial agent, such as a carbapenem, an aminoglycoside, a polymyxin, a glycylcycline, rifampicin or sulbactam.

16. (canceled)

17. (canceled)

18. A pharmaceutical aqueous nanosuspension according to claim 1, obtainable by wet milling a suspension of particulate 4-(4-ethyl-5-fluoro-2-hydroxyphenoxy)-3-fluorobenzamide in water.

19. A pharmaceutical aqueous nanosuspension according to claim 1, obtainable by controlled precipitation of nanoparticles of 4-(4-ethyl-5-fluoro-2-hydroxyphenoxy)-3-fluorobenz amide.

20. (canceled)

21. (canceled)

22. (canceled)

23. A method of treatment of microbial infection comprising administering to a subject a therapeutically effective amount of a pharmaceutical aqueous nanosuspension according to claim 1.

24. A method of treatment according to claim 23 wherein the microbial infection is a human or animal infection by Staphylococcus aureus including multiresistant strains such as methicillin-susceptible Staphylococcus aureus (MSSA), methicillin-resistant Staphylococcus aureus (MRSA), vancomycin-intermediate Staphylococcus aureus (VISA) and vancomycin-resistant Staphylococcus aureus (VRSA) strains, Acinetobacter baumannii, Bacillus anthracis, Chlamydophila pneumoniae, Escherichia coli, Haemophilus influenzae, Helicobacter pylori, Klebsiella pneumoniae, Neisseria meningitidis, Neisseria gonorrhoeae, S. intermedius, P. multocida, B. bronchiseptica, M. haemolytica and A. pleuropneumoniae and also bacteria such as Mycobacterium tuberculosis or other organisms such as Plasmodium falciparum.

25. A process for preparing a pharmaceutical aqueous nanosuspension comprising the steps of:
(a) preparing an aqueous suspension comprising particulate 4-(4-ethyl-5-fluoro-2-hydroxyphenoxy)-3-fluorobenzamide and a stabilizing agent; and
(b) milling the aqueous suspension of step (a) to form nanoparticulate 4-(4-ethyl-5-fluoro-2-hydroxyphenoxy)-3-fluorobenzamide.

26. A process according to claim 25, wherein the stabilizing agent is a surfactant or a surfactant polymer, such as polyvinylpyrrolidone.

27. (canceled)

28. A process according to claim 25, wherein step (b) comprises the steps of adding milling media to the suspension, then placing on a roller mill.

29. A process according to claim 25, wherein the resulting 4-(4-ethyl-5-fluoro-2-hydroxyphenoxy)-3-fluorobenzamide in nanoparticulate form has a volume median diameter (Dv0.5) from about 0.050 μm to about 0.600 μm.

30. A process according to claim 25, wherein the 4-(4-ethyl-5-fluoro-2-hydroxyphenoxy)-3-fluorobenzamide in step (a) is prepared substantially according to the procedure of Example 1 or Example 2.

31. A process according to claim 30, wherein 4-(4-ethyl-5-fluoro-2-hydroxyphenoxy)-3-fluorobenzamide in step (a) has an XRPD pattern substantially as shown in FIG. 1.

32. (canceled)

33. A process for preparing a pharmaceutical aqueous nanosuspension comprising the steps of:
(i) dissolving 4-(4-ethyl-5-fluoro-2-hydroxyphenoxy)-3-fluorobenzamide in an organic solvent;
(ii) adding the solution of step (i) to a solution of a stabilizing agent dissolved in water, whilst maintaining a homogenous solution;
(iii) stirring the solution of step (ii); and
(iv) slowly adding the solution of step (iii) to cool water to form 4-(4-ethyl-5-fluoro-2-hydroxyphenoxy)-3-fluorobenzamide in nanoparticulate form.

34. A process according to claim 33, wherein the organic solvent of step (i) is ethyl acetate.

35. A process according to claim 33, wherein the stabilizing agent is a surfactant or a surfactant polymer such as polyvinylpyrrolidone.

36. (canceled)

37. A process according to claim 33, wherein step (iii) takes place at a temperature of between about 30° C. and about 50° C., suitably about 40° C.

38. A process according to claim 33, wherein step (iv) takes place at a temperature of between about 0° C. and about 5° C., suitably about 2° C.

39. A process according to claim 33, wherein the resulting 4-(4-ethyl-5-fluoro-2-hydroxyphenoxy)-3-fluorobenzamide in nanoparticulate form has a volume median diameter (Dv0.5) from about 0.050 μm to about 0.600 μm.

40. A process according to claim 25, further comprising the step of adding a tonicity modifier such as NaCl, mannitol or glucose.

41. (canceled)

42. A process according to claim 25, wherein the nanosuspension is isotonic.

43. A process according to claim 25, wherein the nanosuspension has osmolality of between about 250 mOsm/kg and about 350 mOsm/kg, for example between about 280 mOsm/kg and about 320 mOsm/kg.

44. (canceled)
